(19) United States
(12) Patent Application Publication
(10) Pub. No.: US 2025/0391575 A1
(43) Pub. Date: Dec. 25, 2025

(54) INFORMATION PROCESSING DEVICE, INFORMATION PROCESSING METHOD, AND NON-TRANSITORY STORAGE MEDIUM

(71) Applicant: JVCKENWOOD Corporation, Yokohama-shi (JP)

(72) Inventor: Ako Suzuki, Yokohama-shi (JP)

(21) Appl. No.: 19/311,007

(22) Filed: Aug. 27, 2025

Related U.S. Application Data
(63) Continuation of application No. PCT/JP2024/006145, filed on Feb. 21, 2024.

(30) Foreign Application Priority Data
Feb. 28, 2023 (JP) 2023-030310
Feb. 28, 2023 (JP) 2023-030406
Feb. 28, 2023 (JP) 2023-030438

(51) Int. Cl. G16H 50/30; G16H 10/60

(52) U.S. Cl. CPC G16H 50/30; G16H 10/60

(57) ABSTRACT

An information processing device includes: a biological information acquiring unit configured to acquire biological information of a subject; an activity calculating unit configured to calculate activity of the subject based on the biological information acquired by the biological information acquiring unit; an emotional level calculating unit configured to calculate emotional level of the subject based on the biological information acquired by the biological information acquiring unit; and a classifying unit configured to classify the biological information based on the activity which is calculated by the activity calculating unit and the emotional level which is calculated by the emotional level calculating unit.

### CROSS-REFERENCE TO RELATED APPLICATIONS

This application is a Continuation of PCT International Application No. PCT/JP2024/006145 filed on Feb. 21, 2024 which claims the benefit of priority from Japanese Patent Applications No. 2023-030310, No. 2023-030406 and No. 2023-030438, filed on Feb. 28, 2023, the entire contents of all of which are incorporated herein by reference.

### FIELD OF THE INVENTION

The present application is related to an information processing device, an information processing method, and a non-transitory storage medium.

### BACKGROUND OF THE INVENTION

In recent years, there is advancement in the technology for measuring brain activity information, and the technology about a brain-machine interface, which represents the interface between the brain and the outside, is becoming more and more realistic. In Japanese Patent Application Laid-open 2008-104528, it is disclosed that pulsation component data is extracted from heart rate data, depth of sleep of the brain is measured based on the pulsation component data, and quality of sleep, in which a sleep state corresponding to REM sleep and non-REM sleep is estimated from the depth of sleep of the brain, is displayed.

However, in Japanese Patent Application Laid-open 2008-104528, there is no suggestion about extracting a phenomenon in a dream of a subject who has a dream in a sleep state to the outside.

### SUMMARY OF THE INVENTION

An information processing device, an information processing method, and a non-transitory storage medium are disclosed.

According to one aspect of the present application, there is provided an information processing device comprising: a biological information acquiring unit configured to acquire biological information of a subject; an activity calculating unit configured to calculate activity of the subject based on the biological information acquired by the biological information acquiring unit; an emotional level calculating unit configured to calculate emotional level of the subject based on the biological information acquired by the biological information acquiring unit; and a classifying unit configured to classify the biological information based on the activity which is calculated by the activity calculating unit and the emotional level which is calculated by the emotional level calculating unit.

According to one aspect of the present application, there is provided an information processing method comprising: acquiring biological information of a subject; calculating activity of the subject based on the biological information; calculating emotional level of the subject based on the biological information; and classifying the biological information based on the activity and the emotional level.

According to one aspect of the present application, there is provided a non-transitory storage medium that stores a program that causes a computer, which operates as an information processing device, to execute: acquiring biological information of a subject; calculating activity of the subject based on the biological information; calculating emotional level of the subject based on the biological information; and classifying the biological information based on the activity and the emotional level.

The above and other objects, features, advantages and technical and industrial significance of this application will be better understood by reading the following detailed description of presently preferred embodiments of the application, when considered in connection with the accompanying drawings.

### BRIEF DESCRIPTION OF THE DRAWINGS

FIG. 1 is a block configuration diagram illustrating an information processing device according to a first embodiment;

FIG. 2 is a graph for explaining physiological characteristics of a biological signal;

FIG. 3 is a schematic diagram for explaining the autonomic nervous system activity;

FIG. 4 is a flowchart for explaining an information processing method according to the first embodiment;

FIG. 5 is a block configuration diagram illustrating an information processing device according to a second embodiment;

FIG. 6 is a flowchart for explaining an information processing method according to the second embodiment;

FIG. 7 is a block configuration diagram illustrating an information processing device according to a third embodiment;

FIG. 8 is a flowchart for explaining an information processing method according to the third embodiment;

FIG. 9 is a block configuration diagram illustrating an information processing device according to a fourth embodiment;

FIG. 10 is a graph for explaining physiological characteristics of a biological signal;

FIG. 11 is a schematic diagram for explaining the autonomic nervous system activity;

FIG. 12 is a flowchart for explaining an information processing method according to the fourth embodiment;

FIG. 13 is a block configuration diagram illustrating an information processing device according to a fifth embodiment;

FIG. 14 is a flowchart for explaining an information processing method according to the fifth embodiment;

FIG. 15 is a block configuration diagram illustrating an information processing device according to a sixth embodiment;

FIG. 16 is a graph for explaining physiological characteristics of a biological signal;

FIG. 17 is a schematic diagram for explaining the autonomic nervous system activity; and

FIG. 18 is a flowchart for explaining an information processing method according to the sixth embodiment.

### DETAILED DESCRIPTION OF THE INVENTION

Exemplary embodiments of an information processing device, an information processing method, and a non-transitory storage medium according to the present application are described in detail with reference to the accompanying drawings. However, the present invention is not limited to the embodiments described below.

### First Embodiment

### Information Processing Device

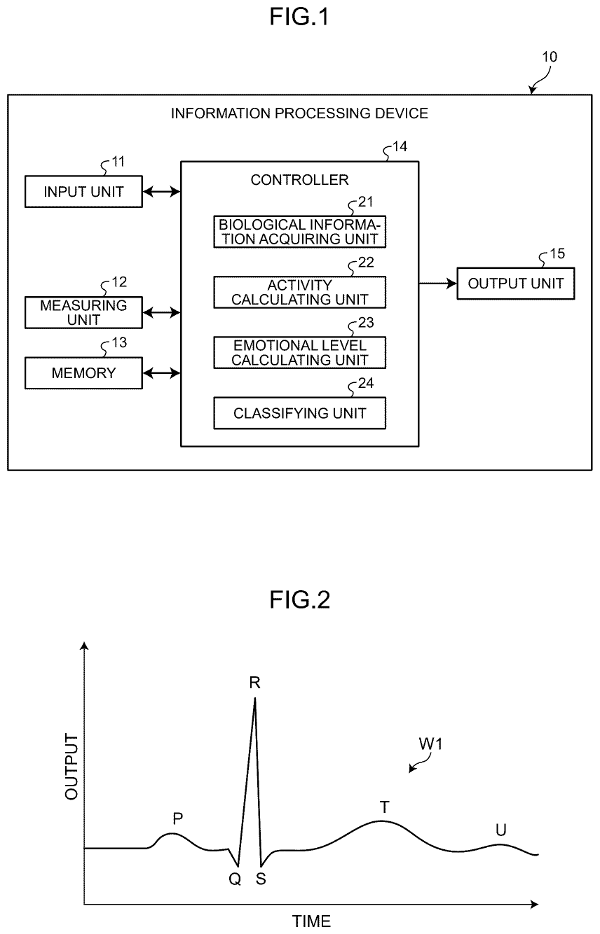

FIG. 1 is a block configuration diagram illustrating an information processing device according to a first embodiment.

As illustrated in FIG. 1, an information processing device 10 enables protecting a phenomenon in a dream recalled by a user (subject) in a sleep state. The information processing device 10 includes an input unit 11, a measuring unit 12, a memory 13, a controller 14, and an output unit 15.

The input unit 11 is connected to the controller 14. The input unit 11 is operable by the user, and is capable of inputting various signals to the controller 14. For example, the input unit 11 inputs, to the controller 14, a start signal for starting an operation of outputting the dream of the user in a sleep state, to an outside, or an end signal for ending the operation of outputting the dream of the user. The input unit 11 can be implemented using, for example, a touch-sensitive panel, or buttons, or switches, or a keyboard.

The measuring unit 12 is connected to the controller 14. Based on a program, the controller 14 provides a measurement signal to the measuring unit 12. Then, based on the measurement signal input from the controller 14, the measuring unit 12 measures biological information of the user.

The measuring unit 12 is a biological sensor that detects the biological information of the user. As long as the biological information of the user can be detected, the biological sensor can be installed at an arbitrary position. Herein, the biological information does not imply permanent information such as the fingerprints, but implies values that vary according to a condition of the user. That is, the biological information represents information related to the autonomic nerves of the user, that is, information that changes in values regardless of an intention of the user.

As the biological information, the measuring unit 12 measures, for example, the brain waves, the cerebral blood value, the heart rate, the respiratory rate, the blood pressure, the body temperature, the amount of perspiration, and the myoelectric current. As the measuring unit 12, for example, a measurement device that performs measurement based on a principle of fMRI (which stands for functional Magnetic Resonance Imaging) or fNIRS (which stands for functional Near-Infrared Spectroscopy), a measurement device in which an invasive electrode is used, or a measurement device that performs measurement using micromachines that are placed inside blood vessels of the brain is able to be used.

Alternatively, the measuring unit 12 can be a pulse wave sensor as the biological sensor. Accordingly, the measuring unit 12 detects pulse waves of the user as the biological information. For example, the pulse wave sensor can be a through-beam photoelectric sensor that includes a light emitting unit and a light receiving unit. In that case, for example, the pulse wave sensor is configured in such a way that the light emitting unit and the light receiving unit face each other across the fingertip of the user; the light receiving unit receives light which has passed through the fingertip; and the pulse waveform is measured based on the fact that the blood flow is higher in proportion to the pressure of the pulse waves. However, the pulse wave sensor is not limited to have the configuration explained above, and can be configured in an arbitrary manner as long as the pulse waves can be detected.

The memory 13 is connected to the controller 14. The memory 13 stores therein a variety of information. In the memory 13, an activity threshold value and an emotional level threshold value, which are to be used during an output process performed by the controller 14, are stored in advance. The activity threshold value is, for example, a preset threshold value of an autonomic nervous system activity, and represents degree of clarity of a phenomenon in a dream who has a dream in a sleep state. From among dreams, those dreams which are story-centered and which can be recalled in detail after waking up are often observed during REM sleep. On the other hand, from among dreams, those dreams which are not story-centered and which are fragmentary are often observed in non-REM sleep. During REM sleep, there is an increase in the autonomic nervous system activity (explained later), and during non-REM sleep, there is a decrease in the autonomic nervous system activity. For that reason, the autonomic nervous system activity serves as a guideline for indicating degree of clarity of phenomenon in a dream of the user who has a dream in a sleep state. The emotional level threshold value is, for example, a preset threshold value of an emotional level, and represents degree of variation in emotions toward the phenomenon in a dream of the user who has a dream in a sleep state.

Meanwhile, the activity threshold value and the emotional level threshold value are not limited to the threshold values as explained above. As a method for estimating a phenomenon in a dream of the user in a sleep state, the following technology is known. For example, a sparse coding theory, which is a method for visualization of transition of recognition from the first visual cortex, is implemented in which, an FMRI activity map of the visual cortex is visualized by a DNN (Deep Neural Network)-CNN (Convolutional Neural Network), simply and locally processed in the primary visual cortex, and then recognized in a stepwise manner in the secondary visual cortex. According to this method, in the brain stimulation and the brain cognition (cognition of having a dream in case of a vision) that is unique to the user, the relationship among the trigger, the recalled image, and the sound, that is, the image data in the brain recalled by the trigger can be obtained according to the biological information of the user.

The phenomenon in a dream of the user who has a dream in the sleep state can be estimated based on image data corresponding to the biological information of the user. The degree of clarity of a phenomenon in a dream can be estimated based on, for example, edges, motion vector, contrast, and resolution with respect to the image data corresponding to the biological information of the user. The activity threshold value is set based on the level of the autonomic nervous system activity of the user. However, alternatively, the activity threshold value can be set based on the level of the image data corresponding to the biological information of the user.

In the first embodiment, the emotional level threshold value is set based on degree of variation in the emotions of the user. The human emotions include feelings such as joy, anger, sorrow, and pleasure, and, for example, can be divided into categories by specific emotions such as amazement, joy, anger, fear, sadness, and disgust. Hence, the emotional level threshold value can be set based on the degree of amazement, the degree of joy, the degree of anger, the degree of sadness, and the degree of disgust.

In the memory 13, a program is stored that enables the controller 14 to perform information processing. The memory 13 is an external storage device such as an HDD (which stands for Hard Disk Drive), or is a memory.

The controller 14 includes a biological information acquiring unit 21, an activity calculating unit 22, an emotional level calculating unit 23, and a classifying unit 24. For example, the controller 14 is configured using an arithmetic circuit such as a CPU (which stands for Central Processing Unit).

The biological information acquiring unit 21 is connected to the measuring unit 12. The biological information acquiring unit 21 controls the measuring unit 12 and causes the measuring unit 12 to detect the biological information of the measuring unit 12. Then, the biological information acquiring unit 21 acquires the biological information of the user measured by the measuring unit 12.

The biological information acquiring unit 21 is connected to the activity calculating unit 22. The activity calculating unit 22 calculates the autonomic nervous system activity based on the biological signal acquired by the biological information acquiring unit 21. Regarding the calculation method implemented by the activity calculating unit 22 to calculate the autonomic nervous system activity, the explanation is given later.

The biological information acquiring unit 21 is connected to the emotional level calculating unit 23. The emotional level calculating unit 23 calculates the emotional level based on the biological signal acquired by the biological information acquiring unit 21. Regarding the calculation method implemented by the emotional level calculating unit 23 to calculate the emotional level, the explanation is given later.

The activity calculating unit 22 and the emotional level calculating unit 23 are connected to the memory 13 and the classifying unit 24. The classifying unit 24 compares the autonomic nervous system activity which is calculated by the activity calculating unit 22 with an activity threshold value stored in the memory 13 to determine whether or not it is possible to perform the output process. Moreover, the classifying unit 24 compares the emotional level which is calculated by the emotional level calculating unit 23 with an emotional level threshold value stored in the memory 13 to determine whether or not it is possible to perform the output process.

More particularly, when the user is in the sleep state and is having a dream and recalling a specific phenomenon in the dream (sensory information), the classifying unit 24 compares the autonomic nervous system activity at that time with the activity threshold value, and accordingly determines whether or not the output process for outputting the phenomenon in the dream to the outside can be performed. In an identical manner, when the user is in the sleep state and is having a dream and recalling a specific phenomenon (sensory information), the classifying unit 24 compares the emotional level at that time with the emotional level threshold value, and accordingly determines whether or not the output process for outputting the phenomenon in the dream to the outside can be performed.

In the first embodiment, when the autonomic nervous system activity of the user is equal to or lower than the activity threshold value, that is, when the user in the sleep state is having a low degree of clarity of the phenomenon in a dream of the user who has a dream, the classifying unit 24 turns off (OFF) a first flag that is set for disabling the output process. Moreover, when the emotional level of the user is equal to or lower than the corresponding emotional level threshold value, that is, when the user in the sleep state is having a low degree of variation in the emotions felt toward the phenomenon in a dream of the user who has a dream in the sleep state, the classifying unit 24 turns off (OFF) a second flag that is set for disabling the output process. When the first flag or the second flag is turned off (OFF), the classifying unit 24 allows implementation of the output process for outputting the phenomenon in the dream to the outside, and classifies that the biological information of the user at that time can be output.

On the other hand, when the autonomic nervous system activity of the user is higher than the activity threshold value, that is, when the user in the sleep state is having a high degree of clarity of the phenomenon in a dream of the user who has a dream, the classifying unit 24 turns on (ON) the first flag that is set for disabling the output process. Moreover, when the emotional level of the user is higher than the corresponding emotional level threshold value, that is, when the user in the sleep state is having a high degree of variation in the emotions felt toward the phenomenon in the dream of the user who has a dream, the classifying unit 24 turns on (ON) the second flag that is set for disabling the output process. When the first flag and the second flag is turned on (ON), the classifying unit 24 disables implementation of the output process for outputting the phenomenon in the dream to the outside, and classifies that the biological information of the user at that time cannot be output.

In that case, multiple relationships between the degree of clarity of the recalled phenomenon in the dream and the autonomic nervous system activity during the sleep state of the user are obtained in advance. Then, regarding the obtained relationships between the degree of clarity of the recalled phenomenon and the autonomic nervous system activity, it is desirable to set the activity threshold value according to the degree of clarity of the phenomenon in the dream which is acceptable to the user as an externally-outputtable phenomenon in the dream. Moreover, multiple emotional levels corresponding to the recalled phenomenon in the dream during the sleep state of the user are obtained in advance. Regarding the obtained emotional levels corresponding to the phenomenon in the dream, it is desirable to set the emotional level threshold value according to the emotional levels of the phenomenon in the dream which is acceptable to the user as an externally-outputtable phenomenon.

The controller 14 is connected to the output unit 15. The output unit 15 transmits to the outside to display a control result by the controller 14, that is, transmits to the outside to display the phenomenon in the dream of the user that is classified to be outputtable by the classifying unit 24. The output unit 15 is a display that displays videos, or is a sound output device that outputs sounds.

### Autonomic Nervous System Activity

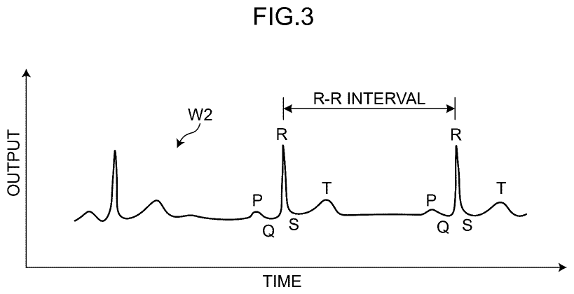

FIG. 2 is a graph for explaining physiological characteristics of a biological signal. FIG. 3 is a schematic diagram for explaining the autonomic nervous system activity. In the explanation of FIGS. 2 and 3, the biological signal is assumed to be electrocardiogram. However, instead of electrocardiogram, the biological signal such as a pulse wave or a brain wave can be used. By a second-order differentiation of pulse waves, a signal corresponding to an R-R interval of electrocardiogram is able to be obtained.

As illustrated in FIG. 2, a waveform W1 representing electrocardiogram includes a P wave, a QRS wave, a T wave, and a U wave. The heart rate is measured by detecting the R wave which represents a peak of the QRS wave as one pulse.

The electrocardiogram is a waveform in which peaks called R-wave appear at regular time intervals. The pulse occurs due to autoignition of pacemaker cells in the sinoatrial node of the heart. Rhythm of the pulse is heavily influenced by the sympathetic nervous system and the parasympathetic nervous system. The sympathetic nervous system enhances the heart activity, while the parasympathetic nervous system suppresses the heart activity. Normally, the sympathetic nervous system and the parasympathetic nervous system act to counterbalance each other. When at rest or in a state close to resting, the parasympathetic nervous system becomes dominant. Normally, when adrenaline is secreted due to the activation of the sympathetic nervous system, the pulse rate increases. On the other hand, when acetylcholine is secreted due to the activation of the parasympathetic nervous system, the pulse rate decreases. Hence, regarding a functional inspection of the autonomic nerve system, it is assumed that checking variability in an R-R interval in the electrocardiogram proves useful.

As illustrated in FIG. 3, in a waveform W2 representing the electrocardiogram, the R-R interval indicates an interval between chronologically continuous R-wave. The heart rate variability is measured by treating the R wave, which represents the peak of the QRS wave, as one pulse. The variability in the interval between the R waves in the electrocardiogram, that is, a fluctuation in the time interval of the R-R interval in FIG. 3 is used as an autonomic nervous system indicator. An appropriateness of using the fluctuation in the time interval of the R-R interval as the autonomic nervous system indicator has been reported in many medical institutions. The fluctuation of the R-R interval increases when at rest and decreases when in stress.

The variability in the R-R interval includes a few types of characteristic fluctuations. One type of fluctuation represents low-frequency component appearing in the vicinity of 1 Hz and is attributed to the variation in the sympathetic nervous system along with the blood pressure feedback control of the blood vessels. Another type of fluctuation indicates the variation occurring in synchronization with breathing, and represents high-frequency component that reflect the respiratory sinus arrhythmia. The high-frequency component reflects direct interference with vagal preganglionic neuron due to the respiratory center, stretch receptor reflex, and baroreceptor reflex of the blood pressure change due to the breathing, and is treated as the parasympathetic nervous system indicator that mainly affects the heart. That is, it can be said that, from among waveform components in which the fluctuation between the R-R waves of the electrocardiogram is measured, power spectrum of the low-frequency component represents the activity of the sympathetic nervous system, and power spectrum of the high-frequency components represent the activity of the parasympathetic nervous system.

The fluctuation of the input electrocardiogram is obtained from a differential value of the R-R interval value. In that case, when the differential values of the R-R intervals do not represent equally spaced time-series data, the activity calculating unit 22 converts those values into equally spaced time-series data using a three-dimensional spline interpolation. The activity calculating unit 22 performs orthogonal transform such as fast Fourier transform with respect to the differential values of the R-R intervals. Thus, the activity calculating unit 22 calculates the power spectrum of the high-frequency components and the power spectrum of the low-frequency components of the differential values of the R-R interval values of the electrocardiogram. The activity calculating unit 22 calculates a sum total of the power spectrum of the high-frequency component as RRHF. Moreover, the activity calculating unit 22 calculates a sum total of the power spectrum of the low-frequency component as RRLF. The activity calculating unit 22 calculates the autonomic nervous system activity using the following equation.

AN=(C1+RRLF)/(C1+RRHF)+C2

In the equation given above, AN represents the autonomic nervous system activity, RRHF represents the sum total of the power spectrum of the high-frequency component, and RRLF represents the sum total of the power spectrum of the low-frequency component. Moreover, C1 and C2 are fixed values defined for suppressing divergence of solutions of the autonomic nervous system activity AN.

The activity calculating unit 22 sets an activity threshold value based on the multiple autonomic nerve activities AN calculated for the user, and stores the activity threshold value in the memory 13.

### Emotional Level

In an identical manner to the autonomic nervous system activity, the emotional level is also calculated based on a biological signal such as the electrocardiogram or the brain wave signal acquired from the user. For example, the emotional level calculating unit 23 calculates the emotional level based on the brain wave signal acquired from the user. More particularly, the emotional level calculating unit 23 extracts α waves and β waves from the brain wave signals. The α waves increase in a relaxed state, and the β waves increase when joy, anger, or nervousness is felt. Hence, the emotional level calculating unit 23 calculates the emotional level according to the extracted β waves/α waves. However, the abovementioned calculation method for calculating the emotional level is only exemplary. Thus, the emotional level calculating unit 23 can calculate the emotional level using the electrocardiogram from among the biological signals, or can calculate the emotional level using the electrocardiogram and the brain wave signals from among the biological signals.

In the memory 13, the emotional level threshold value is stored in advance. Since the emotional level threshold value is different for each individual, the emotional level can be calculated according to the calculation method explained above for calculating the emotional level, and the emotional level threshold value can be set with reference to the values thereof.

### Information Processing Method

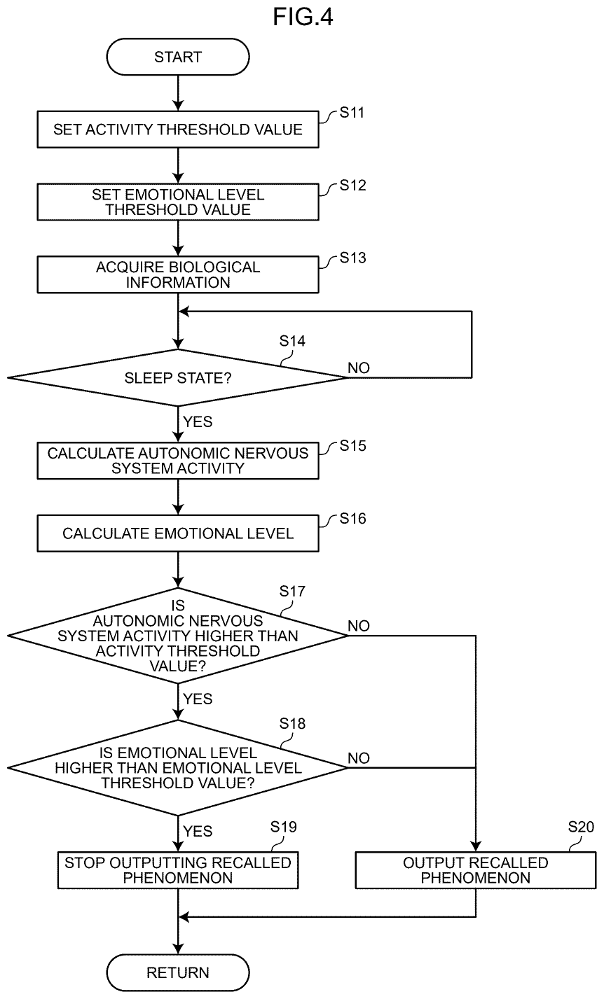

FIG. 4 is a flowchart for explaining an information processing method according to the first embodiment.

As illustrated in FIGS. 1 and 4, at Step S11, the activity threshold value is set. It is desirable to set the activity threshold value separately for each user of the information processing device 10. At Step S12, the emotional level threshold value is set. It is desirable to set the emotional level threshold value separately for each user of the information processing device 10. The processes at Steps S11 and S12 can be performed before the information processing device 10 is used.

After the processes at Steps S11 and S12 are completed, the user of the information processing device 10 goes to sleep. At Step S13, the measuring unit 12 measures the biological information of the user, and the biological information acquiring unit 21 acquires the biological information of the user measured by the measuring unit 12. At Step S14, based on the biological information of the user, the controller 14 determines whether or not the user is in the sleep state. According to the biological information of the user, when the autonomic nervous system activity calculated using the electrocardiogram is determined to match with the autonomic nervous system activity during REM sleep or during non-REM sleep, the controller 14 determines that the user has transitioned into the sleep state. For example, the controller 14 can determine that the user has transitioned into the sleep state based on the brain waves, the electrocardiogram, the pulse waves, the pulse rate, the respiratory rate, and the autonomic nervous system activity as the biological information.

When it is determined that the user is not in the sleep state (No), the controller 14 maintains the present state. On the other hand, when it is determined that the user is in the sleep state (Yes), at Step S15, the activity calculating unit 22 calculates the autonomic nervous system activity based on the biological information of the user acquired by the biological information acquiring unit 21. At Step S16, the emotional level calculating unit 23 calculates the emotional level based on the biological information of the user acquired by the biological information acquiring unit 21. Meanwhile, the process performed by the activity calculating unit 22 and the process performed by the emotional level calculating unit 23 can be performed in reverse order or in a simultaneous manner. At Step S17, the classifying unit 24 determines whether or not the autonomic nervous system activity which is calculated by the activity calculating unit 22 is higher than the activity threshold value of the user stored in the memory 13. When the classifying unit 24 determines that the autonomic nervous system activity of the user is equal to or lower than the corresponding activity threshold value (No), the process proceeds to Step S20.

On the other hand, when the classifying unit 24 determines that the autonomic nervous system activity of the user is higher than the corresponding activity threshold value (Yes), the process proceeds to Step S18. At Step S18, the classifying unit 24 determines whether or not the emotional level calculated by the emotional level calculating unit 23 is higher than the corresponding emotional level threshold value for the user stored in the memory 13. When the classifying unit 24 determines that the emotional level of the user is equal to or lower than the corresponding emotional level threshold value (No), the process proceeds to Step S20. On the other hand, when the classifying unit 24 determines that the emotional level of the user is higher than the corresponding emotional level threshold value (Yes), the process proceeds to Step S19.

That is, when it is determined at Step S17 that the autonomic nervous system activity of the user is equal to or lower than the corresponding activity threshold value and when it is determined at Step S18 that the emotional level of the user is equal to or lower than the corresponding emotional level threshold value, the classifying unit 24 classifies that the output process for outputting, to the outside, the phenomenon in the dream recalled by the user can be performed. Then, the process proceeds to Step S20, and the output unit 15 outputs the biological information of the user to the outside, that is, outputs the phenomenon in the dream recalled by the user to the outside. On the other hand, when it is determined at Step S17 that the autonomic nervous system activity of the user is higher than the corresponding activity threshold value and when it is determined at Step S18 that the emotional level of the user is higher than the corresponding emotional level threshold value, the classifying unit 24 classifies that the output process for outputting the phenomenon in the dream recalled by the user cannot be performed. Then, the process proceeds to Step S19, and the output unit 15 stops outputting the biological information of the user to the outside, that is, stops outputting the phenomenon in the dream recalled by the user to the outside.

### Second Embodiment

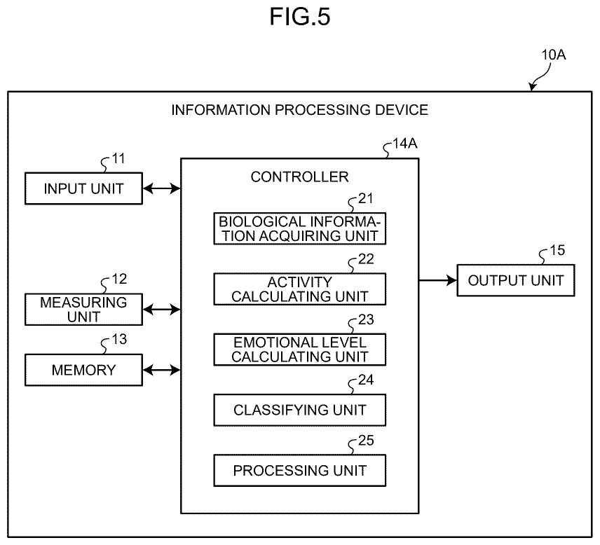

FIG. 5 is a block configuration diagram illustrating an information processing device according to a second embodiment. The constituent elements having identical functions to the functions according to the first embodiment are referred to by the same reference numerals, and their detailed explanation is not given again.

### Information Processing Device

As illustrated in FIG. 5, an information processing device 10A includes the input unit 11, the measuring unit 12, the memory 13, a controller 14A, and the output unit 15. The input unit 11, the measuring unit 12, the memory 13, and the output unit 15 are identical to the first embodiment.

The controller 14A includes the biological information acquiring unit 21, the activity calculating unit 22, the emotional level calculating unit 23, the classifying unit 24, and a processing unit 25. The biological information acquiring unit 21, the activity calculating unit 22, the emotional level calculating unit 23, and the classifying unit 24 are identical to the first embodiment.

The processing unit 25 is connected to the biological information acquiring unit 21 and the classifying unit 24. The processing unit 25 can encrypt or encode the biological information acquired by the biological information acquiring unit 21. More particularly, when the classifying unit 24 classifies the biological information such that the activity is greater than the corresponding activity threshold value and the emotional level is greater than the corresponding emotional level threshold value, the processing unit 25 encrypts or encodes the biological information.

Herein, encryption or encoding is an operation for ensuring that a content of predetermined data is not seen by a third person. In encryption, the original data is subjected to special processing and is converted into different data. In encoding, the original data is substituted according to certain rules and is converted into different data.

The controller 14A is connected to the output unit 15. The output unit 15 transmits, to the outside, and displays the control result obtained by the controller 14A, that is, transmits, to the outside, and displays the phenomenon of the user that are classified by the classifying unit 24. In that case, the biological information classified by the classifying unit 24 to have the activity equal to or lower than the corresponding activity threshold value and the biological information classified by the classifying unit 24 to have the emotional level equal to or lower than the corresponding emotional level threshold value are output by the output unit 15 as they are without any encryption or encoding. Moreover, the biological information classified by the classifying unit 24 to have the activity higher than the corresponding activity threshold value and the biological information classified by the classifying unit 24 to have the emotional level higher than the corresponding emotional level threshold value are output by the output unit 15 after the processing unit 25 has encrypted or encoded the biological information.

### Information Processing Method

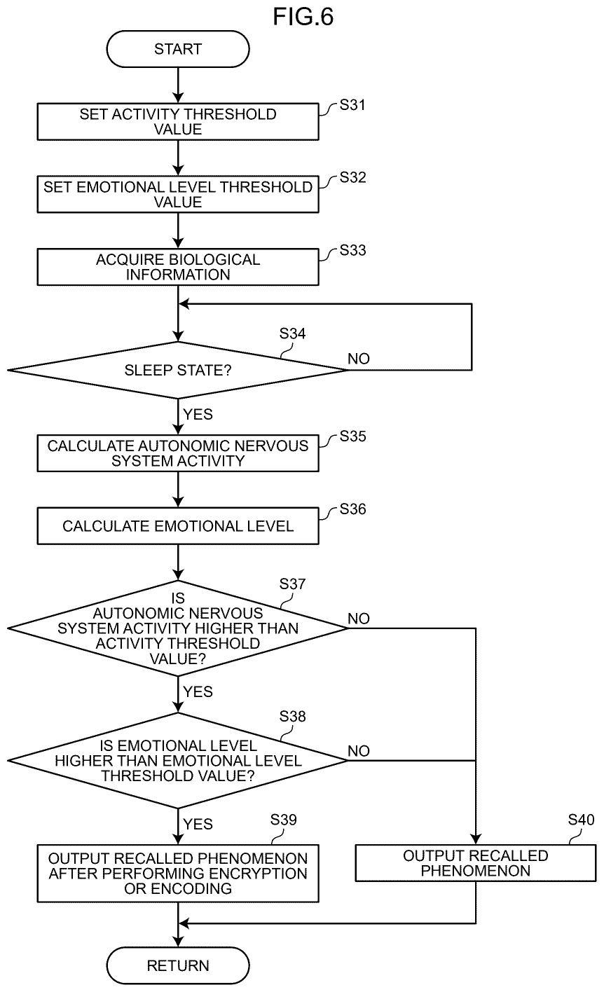

FIG. 6 is a flowchart for explaining an information processing method according to the second embodiment.

As illustrated in FIGS. 5 and 6, at Step S31, the activity threshold value is set. At Step S32, the emotional level threshold value is set. After the processes at Steps S31 and S32 are completed, the user of the information processing device 10A goes to sleep.

At Step S33, the measuring unit 12 measures the biological information of the user, and the biological information acquiring unit 21 acquires the biological information of the user measured by the measuring unit 12. At Step S34, based on the biological information of the user, the controller 14A determines whether or not the user is in the sleep state. When it is determined that the user is not in the sleep state (No), the controller 14A maintains the present state. On the other hand, when it is determined that the user is in the sleep state (Yes), at Step S35, the activity calculating unit 22 calculates the autonomic nervous system activity based on the biological information of the user acquired by the biological information acquiring unit 21. At Step S36, the emotional level calculating unit 23 calculates the emotional level based on the biological information of the user acquired by the biological information acquiring unit 21. At Step S37, the classifying unit 24 determines whether or not the autonomic nervous system activity, which is calculated by the activity calculating unit 22, is higher than the activity threshold value of the user stored in the memory 13. When the classifying unit 24 determines that the autonomic nervous system activity of the user is equal to or lower than the corresponding activity threshold value (No), the process proceeds to Step S40.

When the classifying unit 24 determines that the autonomic nervous system activity of the user is higher than the corresponding activity threshold value (Yes), the process proceeds to Step S38. At Step S38, the classifying unit 24 determines whether or not the emotional level calculated by the emotional level calculating unit 23 is higher than the corresponding emotional level threshold value of the user stored in the memory 13. When the classifying unit 24 determines that the emotional level of the user is equal to or lower than the corresponding emotional level threshold value (No), the process proceeds to Step S40. On the other hand, when the classifying unit 24 determines that the emotional level of the user is higher than the corresponding emotional level threshold value (Yes), the process proceeds to Step S39.

That is, when it is determined at Step S37 that the autonomic nervous system activity of the user is equal to or lower than the corresponding activity threshold value and when it is determined at Step S38 that the emotional level of the user is equal to or lower than the corresponding emotional level threshold value, the classifying unit 24 classifies that the output process for outputting, to the outside, the phenomenon recalled by the user can be performed. Then, the process proceeds to Step S40, and the output unit 15 outputs, without modification, the biological information of the user to the outside, that is, outputs, without modification, the phenomenon recalled by the user to the outside. On the other hand, when it is determined at Step S37 that the autonomic nervous system activity of the user is higher than the corresponding activity threshold value and when it is determined at Step S38 that the emotional level of the user is higher than the corresponding emotional level threshold value, the classifying unit 24 classifies that the output process for outputting, to the outside, the phenomenon recalled by the user can be performed after encryption or encoding is performed. Then, the process proceeds to Step S39, and the output unit 15 outputs the biological information, which has been encrypted or encoded, to the outside, that is, outputs the phenomenon recalled by the user, which have been encrypted or encoded, to the outside.

### Third Embodiment

### Information Processing Device

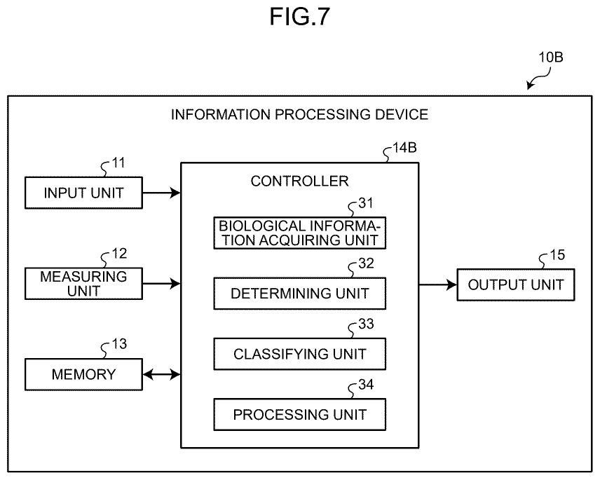

FIG. 7 is a block configuration diagram illustrating an information processing device according to a third embodiment.

As illustrated in FIG. 7, an information processing device 10B enables protection of a phenomenon recalled in a dream of a user (subject) in a sleep state. The information processing device 10B includes the input unit 11, the measuring unit 12, the memory 13, a controller 14B, and the output unit 15.

The input unit 11 is connected to the controller 14B. The input unit 11 is operable by the user, and is capable of inputting various signals to the controller 14B. For example, the input unit 11 inputs, to the controller 14B, a start signal for starting an operation of outputting the dream of the user in a sleep state, to an outside, or an end signal for ending the operation of outputting the dream of the user. The input unit 11 can be implemented using, for example, a touch-sensitive panel, or buttons, or switches, or a keyboard.

The measuring unit 12 is connected to the controller 14B. Based on a program, the controller 14B provides a measurement signal to the measuring unit 12. Then, based on the measurement signal input from the controller 14B, the measuring unit 12 measures biological information of the user.

The measuring unit 12 is a biological sensor that detects the biological information of the user. As long as the biological information of the user can be detected, the biological sensor can be installed at an arbitrary position. Herein, the biological information does not imply permanent information such as the fingerprints, but implies values that vary according to a condition of the user. That is, the biological information represents information related to the autonomic nerves of the user, that is, information that changes in values regardless of an intention of the user.

As the biological information, the measuring unit 12 measures, for example, the brain waves, the cerebral blood value, the heart rate, the respiratory rate, the blood pressure, the body temperature, the amount of perspiration, and the myoelectric current. As the measuring unit 12, for example, a measurement device that performs measurement based on a principle of EMRI (which stands for functional Magnetic Resonance Imaging) or fNIRS (which stands for functional Near-Infrared Spectroscopy), a measurement device in which an invasive electrode is used, or a measurement device that performs measurement using micromachines that are placed inside blood vessels of the brain is able to be used.

Alternatively, the measuring unit 12 can be a pulse wave sensor as the biological sensor. Accordingly, the measuring unit 12 detects pulse waves of the user as the biological information. For example, the pulse wave sensor can be a through-beam photoelectric sensor that includes a light emitting unit and a light receiving unit. In that case, for example, the pulse wave sensor is configured in such a way that the light emitting unit and the light receiving unit face each other across the fingertip of the user; the light receiving unit receives light which has passed through the fingertip; and the pulse waveform is measured based on the fact that the blood flow is higher in proportion to the pressure of the pulse waves. However, the pulse wave sensor is not limited to have the configuration explained above, and can be configured in an arbitrary manner as long as the pulse waves can be detected.

The memory 13 is connected to the controller 14B. The memory 13 stores therein a variety of information. In the memory 13, a computer program is stored that enables the controller 14B to perform information processing. The memory 13 is an external storage device such as an HDD (which stands for Hard Disk Drive), or is a memory.

The controller 14B includes a biological information acquiring unit 31, a determining unit 32, a classifying unit 33, and a processing unit 34. For example, the controller 14B is configured using an arithmetic circuit such as a CPU (which stands for Central Processing Unit).

The biological information acquiring unit 31 is connected to the measuring unit 12. The biological information acquiring unit 31 controls the measuring unit 12 and causes the measuring unit 12 to detect the biological information of the measuring unit 12. Then, the biological information acquiring unit 31 acquires the biological information of the user measured by the measuring unit 12.

The biological information acquiring unit 31 is connected to the determining unit 32. Based on the biological signal acquired by the biological information acquiring unit 31, the determining unit 32 determines a sleep state of the user. More particularly, based on the biological information acquired by the biological information acquiring unit 31, the determining unit 32 determines whether the user is in the REM sleep state or in the non-REM sleep state.

The sleep state majorly involves non-REM sleep and REM sleep. During sleep, a person alternately repeats the two different sleep states of non-REM sleep and REM sleep. Non-REM sleep is believed to be a sleep state in which mainly the brain can be rested, and REM sleep is believed to be a sleep state in which the body can be rested. When a person is in the sleep state, shallow non-REM sleep appears at first, and the sleep becomes deeper with time and transitions into REM sleep. Non-REM sleep shows the following characteristics: the activity of the brain waves decreases and a frequency thereof slows down; and a pulse rate, blood pressure, and breathing becomes stable. REM sleep shows the following characteristics: the brain waves show a similar pattern to a light sleep stage from a sleep onset; and the autonomic nervous system functions, such as a pulse rate, breathing, and blood pressure shows irregular variation. Thus, based on the brain waves, the pulse rate, blood pressure, breathing, and the autonomic nervous system activity as the biological signal acquired by the biological information acquiring unit 31, the determining unit 32 determines whether the user is in the REM sleep state or in the non-REM sleep state.

The determining unit 32 is connected to the classifying unit 33. The classifying unit 33 classifies the biological information according to the sleep state of the user determined by the determining unit 32. More particularly, when the user is in the sleep state and is having a dream and recalling a specific phenomenon (sensory information), based on the biological information at that time, the determining unit 32 determines whether the user is in the REM sleep state or in the non-REM sleep state. The classifying unit 33 classifies the biological information of the user into the biological information corresponding to a case in which the determining unit 32 determines that the user is in the REM sleep state, and into the biological information corresponding to a case in which the determining unit 32 determines that the user is in the non-REM sleep state.

The processing unit 34 is connected to the biological information acquiring unit 31 and the classifying unit 33. The processing unit 34 is capable of encrypting (or encoding) the biological information acquired by the biological information acquiring unit 31. More particularly, the processing unit 34 encrypts the biological information that is classified by the classifying unit 33 into those in the REM sleep state.

Herein, encryption or encoding is an operation for ensuring that a content of predetermined data is not seen by a third person. In encryption, the original data is subjected to special processing and is converted into different data. In encoding, the original data is substituted according to certain rules and is converted into different data.

The controller 14B is connected to the output unit 15. The output unit 15 transmits, to the outside, and displays a control result by the controller 14, that is, transmits, to the outside, and displays the biological information of the user classified by the classifying unit 33 or the phenomenon in the dream based on the biological information. In that case, the output unit 15 outputs the biological information that is classified by the classifying unit 33 into those in the non-REM sleep state as it is without any encryption or encoding. Moreover, the output unit 15 outputs that biological information that is classified by the classifying unit 33 into those in the REM sleep state after the processing unit 34 has encrypted or encoded the biological information.

### Information Processing Method

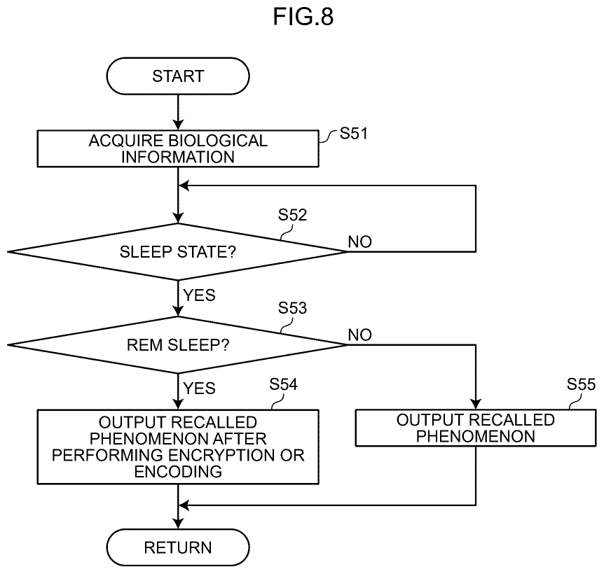

FIG. 8 is a flowchart for explaining an information processing method according to the third embodiment.

As illustrated in FIGS. 7 and 8, at Step S51, the measuring unit 12 measures the biological information of the user, and the biological information acquiring unit 31 acquires the biological information of the user measured by the measuring unit 12. At Step S52, based on the biological information of the user, the controller 14B determines whether or not the user is in the sleep state. According to the biological information of the user, when the autonomic nervous system activity calculated using the electrocardiogram is determined to match with the autonomic nervous system activity during REM sleep or during non-REM sleep, the controller 14B determines that the user has transitioned into the sleep state. For example, based on the brain waves, the electrocardiogram, the pulse waves, the pulse rate, the respiratory rate, and the autonomic nervous system activity as the biological information, the controller 14B can determine that the user has transitioned into the sleep state.

When it is determined that the user is not in the sleep state (No), the controller 14B maintains the present state. On the other hand, when it is determined that the user is in the sleep state (Yes), the process proceeds to Step S53. At Step S53, based on the biological information, the determining unit 32 determines whether or not the user is in the REM sleep state. When the determining unit 32 determines that the user is not in the REM sleep state but is in the non-REM sleep state (No), the process proceeds to Step S55. When the determining unit 32 determines that the user is in the REM sleep state (Yes), the process proceeds to Step S54.

Thus, when the determining unit 32 determines that the user is in the non-REM sleep state, at Step S55, the classifying unit 33 treats non-REM sleep state as the biological information of the user and classifies that the biological information of the user or the phenomenon recalled by the user can be output to the outside without modification, and the output unit 15 outputs the biological information of the user to the outside without modification, that is, outputs the phenomenon recalled by the user to the outside without modification. On the other hand, when the determining unit 32 determines that the user is in the REM sleep state, at Step S54, the classifying unit 33 treats REM sleep state as the biological information of the user and classifies that the biological information of the user or the phenomenon recalled by the user can be output to the outside after encryption (or encoding) is performed, and the output unit 15 outputs the biological information of the user, that is, the phenomenon recalled by the user to the outside after encryption or encoding is performed.

### Fourth Embodiment

### Information Processing Device

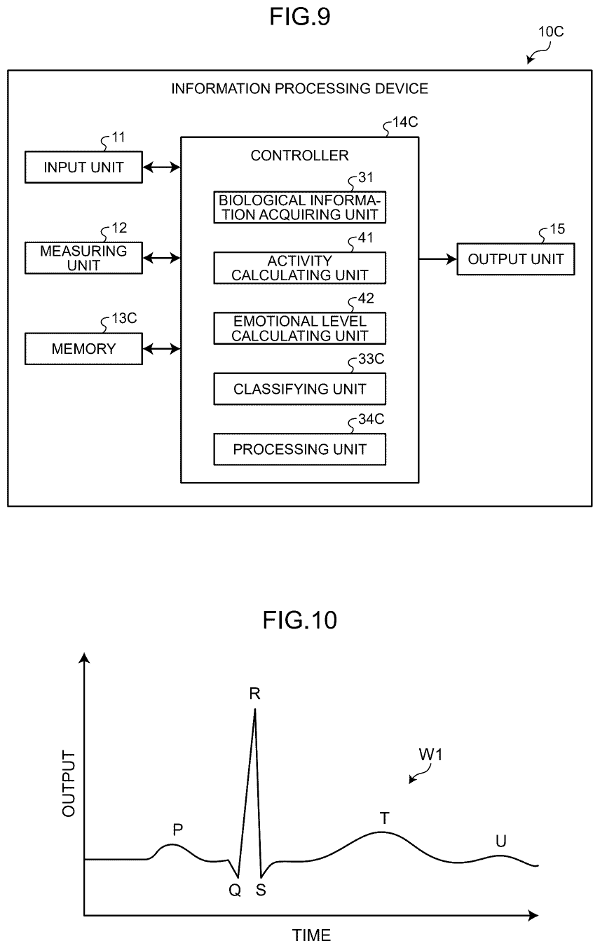

FIG. 9 is a block configuration diagram illustrating an information processing device according to a fourth embodiment. The constituent elements having identical functions to the functions according to the embodiments described above are referred to by the same reference numerals, and their detailed explanation is not given again.

FIG. 9 is a block diagram illustrating the information processing device according to the fourth embodiment.

As illustrated in FIG. 9, an information processing device 10C includes the input unit 11, the measuring unit 12, a memory 13C, a controller 14C, and the output unit 15. The input unit 11, the measuring unit 12, and the output unit 15 are identical to the third embodiment.

The memory 13C is connected to the controller 14C. The memory 13C stores therein a variety of information. In the memory 13C, an activity threshold value and an emotional level threshold value, which are to be used during an output process performed by the controller 14C, are stored in advance. The activity threshold value is, for example, a preset threshold value of an autonomic nervous system activity, and represents degree of clarity of a phenomenon in a dream who has a dream in a sleep state. From among dreams, those dreams which are story-centered and which can be recalled in detail after waking up are often observed during REM sleep. On the other hand, from among dreams, those dreams which are not story-centered and which are fragmentary are often observed in non-REM sleep. During REM sleep, there is an increase in the autonomic nervous system activity (explained later), and during non-REM sleep, there is a decrease in the autonomic nervous system activity. For that reason, the autonomic nervous system activity serves as a guideline for indicating degree of clarity of phenomenon in a dream of the user who has a dream in a sleep state. The emotional level threshold value is, for example, a preset threshold value of emotional level, and represents degree of variation in emotions toward the phenomenon in a dream of the user who has a dream in a sleep state.

Meanwhile, the activity threshold value and the emotional level threshold value are not limited to the threshold values as explained above. As a method for estimating a phenomenon in a dream of the user in a sleep state, the following technology is known. For example, a sparse coding theory, which is a method for visualization of transition of recognition from the first visual cortex, is implemented in which, an FMRI activity map of the visual cortex is visualized by a DNN (Deep Neural Network)-CNN (Convolutional Neural Network), simply and locally processed in the primary visual cortex, and then recognized in a stepwise manner in the secondary visual cortex. According to this method, in the brain stimulation and the brain cognition (cognition of having a dream in case of a vision) that is unique to the user, the relationship among the trigger, the recalled image, and the sound, that is, the image data in the brain recalled by the trigger can be obtained according to the biological information of the user.

The phenomenon in a dream of the user who has a dream in the sleep state can be estimated based on image data corresponding to the biological information of the user. The degree of clarity of a phenomenon in a dream can be estimated based on, for example, edges, motion vector, contrast, and resolution with respect to the image data corresponding to the biological information of the user. The activity threshold value is set based on the level of the autonomic nervous system activity of the user. However, alternatively, the activity threshold value can be set based on the level of the image data corresponding to the biological information of the user.

In the fourth embodiment, the emotional level threshold value is set based on degree of variation in the emotions of the user. The human emotions include feelings such as joy, anger, sorrow, and pleasure, and, for example, can be divided into categories by specific emotions such as amazement, joy, anger, fear, sadness, and disgust. Hence, the emotional level threshold value can be set based on the degree of amazement, the degree of joy, the degree of anger, the degree of sadness, and the degree of disgust.

The controller 14C includes the biological information acquiring unit 31, an activity calculating unit 41, an emotional level calculating unit 42, a classifying unit 33C, and a processing unit 34C. The biological information acquiring unit 31 is identical to the third embodiment. The activity calculating unit 41 and the emotional level calculating unit 42 collectively function as the determining unit 32 according to the third embodiment.

The biological information acquiring unit 31 is connected to the measuring unit 12. The biological information acquiring unit 31 controls the measuring unit 12 and causes the measuring unit 12 to detect the biological information of the measuring unit 12. Then, the biological information acquiring unit 31 acquires the biological information of the user measured by the measuring unit 12.

The biological information acquiring unit 31 is connected to the activity calculating unit 41. The activity calculating unit 41 calculates the autonomic nervous system activity based on the biological signal acquired by the biological information acquiring unit 31. Regarding the calculation method implemented by the activity calculating unit 41 to calculate the autonomic nervous system activity, the explanation is given later.

The biological information acquiring unit 31 is connected to the emotional level calculating unit 42. The emotional level calculating unit 42 calculates the emotional level based on the biological signal acquired by the biological information acquiring unit 31. Regarding the calculation method implemented by the emotional level calculating unit 42 to calculate the emotional level, the explanation is given later.

The activity calculating unit 41 and the emotional level calculating unit 42 are connected to the memory 13C and the classifying unit 33C. The classifying unit 33C compares the autonomic nervous system activity which is calculated by the activity calculating unit 41 with an activity threshold value stored in the memory 13C to determine whether or not it is possible to perform the output process. Moreover, the classifying unit 33C compares the emotional level which is calculated by the emotional level calculating unit 42 with an emotional level threshold value stored in the memory 13C to determine whether or not it is possible to perform the output process.

More particularly, when the user is in the sleep state and is having a dream and recalling a specific phenomenon in the dream (sensory information), the classifying unit 33C compares the autonomic nervous system activity at that time with the activity threshold value, and accordingly determines whether or not the output process for outputting the phenomenon in the dream to the outside can be performed. In an identical manner, when the user is in the sleep state and is having a dream and recalling a specific phenomenon (sensory information), the classifying unit 33C compares the emotional level at that time with the emotional level threshold value, and accordingly determines whether or not the output process for outputting the phenomenon in the dream to the outside can be performed.

In the fourth embodiment, when the autonomic nervous system activity of the user is equal to or lower than the activity threshold value, that is, when the user in the sleep state is having a low degree of clarity of the phenomenon in a dream of the user who has a dream, the classifying unit 33C turns off (OFF) a first flag that is set for disabling the output process. Moreover, when the emotional level of the user is equal to or lower than the corresponding emotional level threshold value, that is, when the user in the sleep state is having a low degree of variation in the emotions felt toward the phenomenon in a dream of the user who has a dream in the sleep state, the classifying unit 33C turns off (OFF) a second flag that is set for disabling the output process. When the first flag or the second flag is turned off (OFF), the classifying unit 33C allows implementation of the output process for outputting the phenomenon in the dream to the outside, and classifies that the biological information of the user at that time can be output.

On the other hand, when the autonomic nervous system activity of the user is higher than the activity threshold value, that is, when the user in the sleep state is having a high degree of clarity of the phenomenon in a dream of the user who has a dream, the classifying unit 33C turns on (ON) the first flag that is set for disabling the output process. Moreover, when the emotional level of the user is higher than the corresponding emotional level threshold value, that is, when the user in the sleep state is having a high degree of variation in the emotions felt toward the phenomenon in the dream of the user who has a dream, the classifying unit 33C turns on (ON) the second flag that is set for disabling the output process. When the first flag and the second flag is turned on (ON), the classifying unit 33C disables implementation of the output process for outputting the phenomenon in the dream to the outside, and classifies that the biological information of the user at that time cannot be output.

In that case, multiple relationships between the degree of clarity of the recalled phenomenon in the dream and the autonomic nervous system activity during the sleep state of the user are obtained in advance. Then, regarding the obtained relationships between the degree of clarity of the recalled phenomenon and the autonomic nervous system activity, it is desirable to set the activity threshold value according to the degree of clarity of the phenomenon in the dream which is acceptable to the user as an externally-outputtable phenomenon in the dream. Moreover, multiple emotional levels corresponding to the recalled phenomenon in the dream during the sleep state of the user are obtained in advance. Regarding the obtained emotional levels corresponding to the phenomenon in the dream, it is desirable to set the emotional level threshold value according to the emotional levels of the phenomenon in the dream which is acceptable to the user as an externally-outputtable phenomenon.

### Autonomic Nervous System Activity

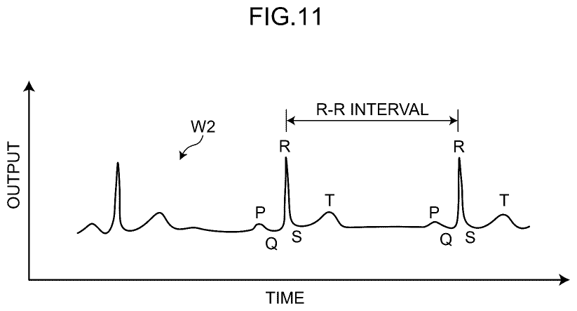

FIG. 10 is a graph for explaining physiological characteristics of a biological signal. FIG. 11 is a schematic diagram for explaining the autonomic nervous system activity. In the explanation of FIGS. 10 and 11, the biological signal is assumed to be electrocardiogram. However, instead of electrocardiogram, the biological signal such as a brain wave can be used. By a second-order differentiation of pulse waves, a signal corresponding to an R-R interval of electrocardiogram is able to be obtained.

As illustrated in FIG. 10, a waveform W1 representing electrocardiogram includes a P wave, a QRS wave, a T wave, and a U wave. The heart rate is measured by detecting the R wave which represents a peak of the QRS wave as one pulse.

The electrocardiogram is a waveform in which peaks called R-wave appear at regular time intervals. The pulse occurs due to autoignition of pacemaker cells in the sinoatrial node of the heart. Rhythm of the pulse is heavily influenced by the sympathetic nervous system and the parasympathetic nervous system. The sympathetic nervous system enhances the heart activity, while the parasympathetic nervous system suppresses the heart activity. Normally, the sympathetic nervous system and the parasympathetic nervous system act to counterbalance each other. When at rest or in a state close to resting, the parasympathetic nervous system becomes dominant. Normally, when adrenaline is secreted due to the activation of the sympathetic nervous system, the pulse rate increases. On the other hand, when acetylcholine is secreted due to the activation of the parasympathetic nervous system, the pulse rate decreases. Hence, regarding a functional inspection of the autonomic nerve system, it is assumed that checking variability in an R-R interval in the electrocardiogram proves useful.

As illustrated in FIG. 11, in a waveform W2 representing the electrocardiogram, the R-R interval indicates an interval between chronologically continuous R-wave. The heart rate variability is measured by treating the R wave, which represents the peak of the QRS wave, as one pulse. The variability in the interval between the R waves in the electrocardiogram, that is, a fluctuation in the time interval of the R-R interval in FIG. 3 is used as an autonomic nervous system indicator. An appropriateness of using the fluctuation in the time interval of the R-R interval as the autonomic nervous system indicator has been reported in many medical institutions. The fluctuation of the R-R interval increases when at rest and decreases when in stress.

The variability in the R-R interval includes a few types of characteristic fluctuations. One type of fluctuation represents low-frequency component appearing in the vicinity of 1 Hz and is attributed to the variation in the sympathetic nervous system along with the blood pressure feedback control of the blood vessels. Another type of fluctuation indicates the variation occurring in synchronization with breathing, and represents high-frequency component that reflect the respiratory sinus arrhythmia. The high-frequency component reflects direct interference with vagal preganglionic neuron due to the respiratory center, stretch receptor reflex, and baroreceptor reflex of the blood pressure change due to the breathing, and is treated as the parasympathetic nervous system indicator that mainly affects the heart. That is, it can be said that, from among waveform components in which the fluctuation between the R-R waves of the electrocardiogram is measured, power spectrum of the low-frequency component represents the activity of the sympathetic nervous system, and power spectrum of the high-frequency components represent the activity of the parasympathetic nervous system.

The fluctuation of the input electrocardiogram is obtained from a differential value of the R-R interval value. In that case, when the differential values of the R-R intervals do not represent equally spaced time-series data, the activity calculating unit 22 converts those values into equally spaced time-series data using a three-dimensional spline interpolation. The activity calculating unit 22 performs orthogonal transform such as fast Fourier transform with respect to the differential values of the R-R intervals. Thus, the activity calculating unit 22 calculates the power spectrum of the high-frequency components and the power spectrum of the low-frequency components of the differential values of the R-R interval values of the electrocardiogram. The activity calculating unit 22 calculates a sum total of the power spectrum of the high-frequency component as RRHF. Moreover, the activity calculating unit 22 calculates a sum total of the power spectrum of the low-frequency component as RRLF. The activity calculating unit 22 calculates the autonomic nervous system activity using the following equation.

AN=(C1+RRLF)/(C1+RRHF)+C2

In the equation given above, AN represents the autonomic nervous system activity, RRHF represents the sum total of the power spectrum of the high-frequency component, and RRLF represents the sum total of the power spectrum of the low-frequency component. Moreover, C1 and C2 are fixed values defined for suppressing divergence of solutions of the autonomic nervous system activity AN.

The activity calculating unit 41 sets an activity threshold value based on the multiple autonomic nerve activities AN calculated for the user, and stores the activity threshold value in the memory 13C.

### Emotional Level

In an identical manner to the autonomic nervous system activity, the emotional level is also calculated based on a biological signal such as the electrocardiogram or the brain wave signal acquired from the user. For example, the emotional level calculating unit 23 calculates the emotional level based on the brain wave signal acquired from the user. More particularly, the emotional level calculating unit 23 extracts α waves and β waves from the brain wave signals. The α waves increase in a relaxed state, and the β waves increase when joy, anger, or nervousness is felt. Hence, the emotional level calculating unit 42 calculates the emotional level according to the extracted β waves/α waves. However, the abovementioned calculation method for calculating the emotional level is only exemplary. Thus, the emotional level calculating unit 42 can calculate the emotional level using the electrocardiogram from among the biological signals, or can calculate the emotional level using the electrocardiogram and the brain wave signals from among the biological signals.

In the memory 13C, the emotional level threshold value is stored in advance. Since the emotional level threshold value is different for each individual, the emotional level can be calculated according to the calculation method explained above for calculating the emotional level, and the emotional level threshold value can be set with reference to the values thereof.

### Information Processing Method

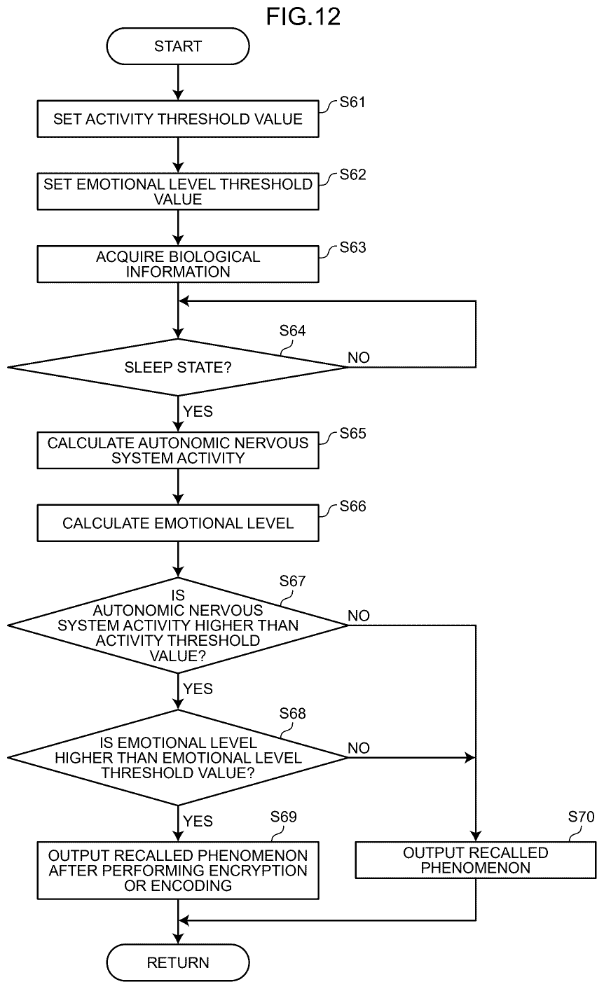

FIG. 12 is a flowchart for explaining an information processing method according to the fourth embodiment.

As illustrated in FIGS. 9 and 12, at Step S61, the activity threshold value is set. It is desirable to set the activity threshold value separately for each user of the information processing device 10C. At Step S12, the emotional level threshold value is set. It is desirable to set the emotional level threshold value separately for each user of the information processing device 10C. The processes at Steps S61 and S62 can be performed before the information processing device 10C is used.

After the operations at Steps S61 and S62 are completed, the user of the information processing device 10C goes to sleep. At Step S63, the measuring unit 12 measures the biological information of the user, and the biological information acquiring unit 31 acquires the biological information of the user measured by the measuring unit 12. At Step S64, based on the biological information of the user, the controller 14C determines whether or not the user is in the sleep state. According to the biological information of the user, when the autonomic nervous system activity calculated using the electrocardiogram is determined to match with the autonomic nervous system activity during REM sleep or during non-REM sleep, the controller 14C determines that the user has transitioned into the sleep state. For example, the controller 14 can determine that the user has transitioned into the sleep state based on the brain waves, the electrocardiogram, the pulse waves, the pulse rate, the respiratory rate, and the autonomic nervous system activity as the biological information. At Step S65, based on the biological information of the user, the controller 14C can determine whether or not the user is in the REM sleep state or in the non-REM sleep state.

When it is determined that the user is not in the sleep state (No), the controller 14C maintains the present state. On the other hand, when it is determined that the user is in the sleep state (Yes), at Step S65, the activity calculating unit 41 calculates the autonomic nervous system activity based on the biological information of the user acquired by the biological information acquiring unit 31. At Step S66, the emotional level calculating unit 42 calculates the emotional level based on the biological information of the user acquired by the biological information acquiring unit 31. Meanwhile, the process performed by the activity calculating unit 41 and the process performed by the emotional level calculating unit 42 can be performed in reverse order or in a simultaneous manner. At Step S67, the classifying unit 33C determines whether or not the autonomic nervous system activity which is calculated by the activity calculating unit 41 is higher than the activity threshold value of the user stored in the memory 13C. When the classifying unit 33C determines that the autonomic nervous system activity of the user is equal to or lower than the corresponding activity threshold value (No), the process proceeds to Step S70.

On the other hand, when the classifying unit 33C determines that the autonomic nervous system activity of the user is higher than the corresponding activity threshold value (Yes), the process proceeds to Step S18. At Step S68, the classifying unit 33C determines whether or not the emotional level calculated by the emotional level calculating unit 42 is higher than the corresponding emotional level threshold value for the user stored in the memory 13C. When the classifying unit 33C determines that the emotional level of the user is equal to or lower than the corresponding emotional level threshold value (No), the process proceeds to Step S70. On the other hand, when the classifying unit 33C determines that the emotional level of the user is higher than the corresponding emotional level threshold value (Yes), the process proceeds to Step S69.

That is, when it is determined at Step S67 that the autonomic nervous system activity of the user is equal to or lower than the corresponding activity threshold value and when it is determined at Step S68 that the emotional level of the user is equal to or lower than the corresponding emotional level threshold value, the classifying unit 33C classifies that the biological information of the user or the phenomenon recalled by the user can be output without modification. Then, the system control proceeds to Step S70, and the output unit 15 outputs, without modification, the biological information of the user to the outside, that is, outputs, without modification, the phenomenon recalled by the user to the outside. On the other hand, when it is determined at Step S67 that the autonomic nervous system activity of the user is higher than the corresponding activity threshold value and when it is determined at Step S68 that the emotional level of the user is higher than the corresponding emotional level threshold value, the classifying unit 33C classifies that the biological information of the user or the recalled phenomenon can be output after the processing unit 34C has encrypted (or encoded) the biological information of the user or the recalled phenomenon. Then, process proceeds to Step S69, and the output unit 15 outputs the biological information, which has been encrypted or encoded, to the outside, that is, outputs the phenomenon recalled by the user, which have been encrypted or encoded, to the outside.

### Fifth Embodiment

### Information Processing Device

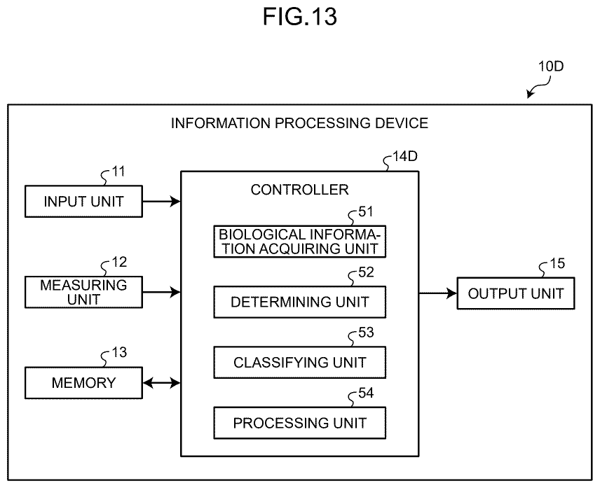

FIG. 13 is a block configuration diagram illustrating an information processing device according to a fifth embodiment.

As illustrated in FIG. 13, an information processing device 10D enables protection of a phenomenon recalled in a dream of a user (subject) in a sleep state. The information processing device 10D includes the input unit 11, the measuring unit 12, the memory 13, a controller 14D, and the output unit 15.

The input unit 11 is connected to the controller 14D. The input unit 11 is operable by the user, and is capable of inputting various signals to the controller 14D. For example, the input unit 11 inputs, to the controller 14D, a start signal for starting an operation of outputting the dream of the user in a sleep state, to an outside, or an end signal for ending the operation of outputting the dream of the user. The input unit 11 can be implemented using, for example, a touch-sensitive panel, or buttons, or switches, or a keyboard.

The measuring unit 12 is connected to the controller 14D. Based on a program, the controller 14D provides a measurement signal to the measuring unit 12. Then, based on the measurement signal input from the controller 14D, the measuring unit 12 measures biological information of the user.

The measuring unit 12 is a biological sensor that detects the biological information of the user. As long as the biological information of the user can be detected, the biological sensor can be installed at an arbitrary position. Herein, the biological information does not imply permanent information such as the fingerprints, but implies values that vary according to a condition of the user. That is, the biological information represents information related to the autonomic nerves of the user, that is, information that changes in values regardless of an intention of the user.

As the biological information, the measuring unit 12 measures, for example, the brain waves, the cerebral blood value, the heart rate, the respiratory rate, the blood pressure, the body temperature, the amount of perspiration, and the myoelectric current. As the measuring unit 12, for example, a measurement device that performs measurement based on a principle of MRI (which stands for functional Magnetic Resonance Imaging) or fNIRS (which stands for functional Near-Infrared Spectroscopy), a measurement device in which an invasive electrode is used, or a measurement device that performs measurement using micromachines that are placed inside blood vessels of the brain is able to be used.

Alternatively, the measuring unit 12 can be a pulse wave sensor as the biological sensor. Accordingly, the measuring unit 12 detects pulse waves of the user as the biological information. For example, the pulse wave sensor can be a through-beam photoelectric sensor that includes a light emitting unit and a light receiving unit. In that case, for example, the pulse wave sensor is configured in such a way that the light emitting unit and the light receiving unit face each other across the fingertip of the user; the light receiving unit receives light which has passed through the fingertip; and the pulse waveform is measured based on the fact that the blood flow is higher in proportion to the pressure of the pulse waves. However, the pulse wave sensor is not limited to have the configuration explained above, and can be configured in an arbitrary manner as long as the pulse waves can be detected.

The memory 13 is connected to the controller 14D. The memory 13 stores therein a variety of information. In the memory 13, a computer program is stored that enables the controller 14D to perform information processing. The memory 13 is an external storage device such as an HDD (which stands for Hard Disk Drive), or is a memory.

The controller 14D includes a biological information acquiring unit 51, a determining unit 52, a classifying unit 53, and a processing unit 54. For example, the controller 14D is configured using an arithmetic circuit such as a CPU (which stands for Central Processing Unit).

The biological information acquiring unit 51 is connected to the measuring unit 12. The biological information acquiring unit 51 controls the measuring unit 12 and causes the measuring unit 12 to detect the biological information of the measuring unit 12. Then, the biological information acquiring unit 51 acquires the biological information of the user measured by the measuring unit 12.

The biological information acquiring unit 51 is connected to the determining unit 52. Based on the biological signal acquired by the biological information acquiring unit 51, the determining unit 52 determines a sleep state of the user. More particularly, based on the biological information acquired by the biological information acquiring unit 51, the determining unit 52 determines whether the user is in the REM sleep state or in the non-REM sleep state.

The sleep state majorly involves non-REM sleep and REM sleep. During sleep, a person alternately repeats the two different sleep states of non-REM sleep and REM sleep. Non-REM sleep is believed to be a sleep state in which mainly the brain can be rested, and REM sleep is believed to be a sleep state in which the body can be rested. When a person is in the sleep state, shallow non-REM sleep appears at first, and the sleep becomes deeper with time and transitions into REM sleep. Non-REM sleep shows the following characteristics: the activity of the brain waves decreases and a frequency thereof slows down; and a pulse rate, blood pressure, and breathing becomes stable. REM sleep shows the following characteristics: the brain waves show a similar pattern to a light sleep stage from a sleep onset; and the autonomic nervous system functions, such as a pulse rate, breathing, and blood pressure shows irregular variation. Thus, based on the brain waves, the pulse rate, blood pressure, breathing, and the autonomic nervous system activity as the biological signal acquired by the biological information acquiring unit 51, the determining unit 52 determines whether the user is in the REM sleep state or in the non-REM sleep state.

The determining unit 52 is connected to the classifying unit 53. The classifying unit 53 classifies the biological information according to the sleep state of the user determined by the determining unit 52. More particularly, when the user is in the sleep state and is having a dream and recalling a specific phenomenon (sensory information), based on the biological information at that time, the determining unit 52 determines whether the user is in the REM sleep state or in the non-REM sleep state. The classifying unit 53 classifies the biological information of the user into the biological information corresponding to a case in which the determining unit 52 determines that the user is in the REM sleep state, and into the biological information corresponding to a case in which the determining unit 52 determines that the user is in the non-REM sleep state.

The processing unit 54 is connected to the biological information acquiring unit 51 and the classifying unit 53. The processing unit 54 is capable of compressing the biological information acquired by the biological information acquiring unit 51. More particularly, the processing unit 54 compresses the biological information that is classified by the classifying unit 53 into those in the REM sleep state.

Herein, the compression is a process for ensuring that a content of predetermined data is not seen by a third person. In the compression, substantial nature (volume of information) of predetermined data is retained as much as possible, and data volume is reduced by converting the original data into different data. Examples of the compression format include, but are not limited to, the ZIP format, the LZH format, and the RAR format.

The controller 14D is connected to the output unit 15. The output unit 15 transmits, to the outside, and displays a control result by the controller 14D, that is, transmits, to the outside, and displays the biological information of the user classified by the classifying unit 53 or the phenomenon in the dream based on the biological information. In that case, the output unit 15 outputs the biological information that is classified by the classifying unit 53 into those in the non-REM sleep state as it is without the compression. Moreover, the output unit 15 outputs that biological information that is classified by the classifying unit 53 into those in the REM sleep state after the processing unit 54 has compressed the biological information.

### Information Processing Method

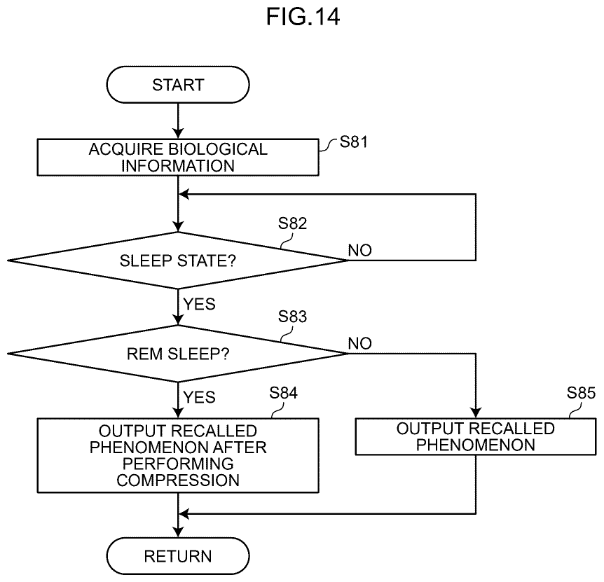

FIG. 14 is a flowchart for explaining an information processing method according to the fifth embodiment.

As illustrated in FIGS. 13 and 14, at Step S81, the measuring unit 12 measures the biological information of the user, and the biological information acquiring unit 51 acquires the biological information of the user measured by the measuring unit 12. At Step S82, based on the biological information of the user, the controller 14D determines whether or not the user is in the sleep state. According to the biological information of the user, when the autonomic nervous system activity calculated using the electrocardiogram is determined to match with the autonomic nervous system activity during REM sleep or during non-REM sleep, the controller 14D determines that the user has transitioned into the sleep state. For example, based on the brain waves, the electrocardiogram, the pulse waves, the pulse rate, the respiratory rate, and the autonomic nervous system activity as the biological information, the controller 14D can determine that the user has transitioned into the sleep state.

When it is determined that the user is not in the sleep state (No), the controller 14D maintains the present state. On the other hand, when it is determined that the user is in the sleep state (Yes), the system control proceeds to Step S83. At Step S83, based on the biological information, the determining unit 52 determines whether or not the user is in the REM sleep state. When the determining unit 52 determines that the user is not in the REM sleep state but is in the non-REM sleep state (No), the process proceeds to Step S85. When the determining unit 52 determines that the user is in the REM sleep state (Yes), the process proceeds to Step S84.

Thus, when the determining unit 52 determines that the user is in the non-REM sleep state, at Step S85, the classifying unit 53 treats the non-REM sleep state as the biological information of the user and classifies that the biological information of the user or the phenomenon recalled by the user can be output to the outside without modification, and the output unit 15 outputs the biological information of the user to the outside without modification, that is, outputs the phenomenon recalled by the user to the outside without modification. On the other hand, when the determining unit 52 determines that the user is in the REM sleep state, at Step S84, the classifying unit 53 treats the REM sleep state as the biological information of the user and classifies that the biological information of the user or the phenomenon recalled by the user can be output to the outside after the compression thereof has been performed, and the output unit 15 outputs the biological information of the user, that is, the phenomenon recalled by the user to the outside after the compression thereof has been performed.

### Sixth Embodiment

### Information Processing Device

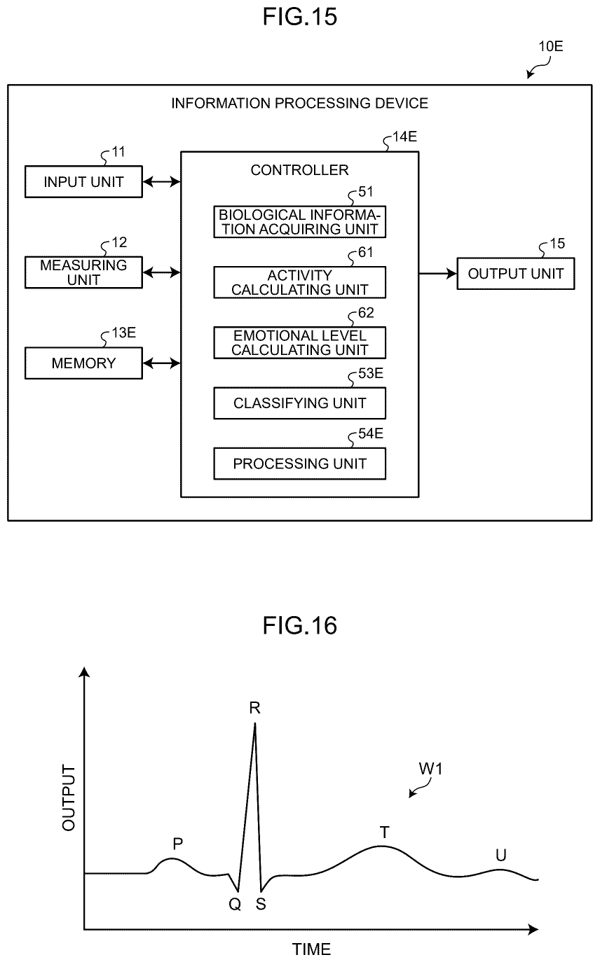

FIG. 15 is a block configuration diagram illustrating an information processing device according to a sixth embodiment. The constituent elements having identical functions to the functions according to the embodiments described above are referred to by the same reference numerals, and their detailed explanation is not given again.

FIG. 15 is a block configuration diagram illustrating the information processing device according to the sixth embodiment.

As illustrated in FIG. 15, an information processing device 10E includes the input unit 11, the measuring unit 12, a memory 13E, a controller 14E, and the output unit 15. The input unit 11, the measuring unit 12, and the output unit 15 are identical to the fifth embodiment.

The memory 13E is connected to the controller 14E. The memory 13E stores therein a variety of information. In the memory 13E, an activity threshold value and an emotional level threshold value, which are to be used during an output operation performed by the controller 14E, are stored in advance. The activity threshold value is, for example, a preset threshold value of an autonomic nervous system activity, and represents degree of clarity of a phenomenon in a dream who has a dream in a sleep state. From among dreams, those dreams which are story-centered and which can be recalled in detail after waking up are often observed during REM sleep. On the other hand, from among dreams, those dreams which are not story-centered and which are fragmentary are often observed in non-REM sleep. During REM sleep, there is an increase in the autonomic nervous system activity (explained later), and during non-REM sleep, there is a decrease in the autonomic nervous system activity. For that reason, the autonomic nervous system activity serves as a guideline for indicating degree of clarity of phenomenon in a dream of the user who has a dream in a sleep state. The emotional level threshold value is, for example, a preset threshold value of emotional level, and represents degree of variation in emotions toward the phenomenon in a dream of the user who has a dream in a sleep state.

Meanwhile, the activity threshold value and the emotional level threshold value are not limited to the threshold values as explained above. As a method for estimating a phenomenon in a dream of the user in a sleep state, the following technology is known. For example, a sparse coding theory, which is a method for visualization of transition of recognition from the first visual cortex, is implemented in which, an FMRI activity map of the visual cortex is visualized by a DNN (Deep Neural Network)-CNN (Convolutional Neural Network), simply and locally processed in the primary visual cortex, and then recognized in a stepwise manner in the secondary visual cortex. According to this method, in the brain stimulation and the brain cognition (cognition of having a dream in case of a vision) that is unique to the user, the relationship among the trigger, the recalled image, and the sound, that is, the image data in the brain recalled by the trigger can be obtained according to the biological information of the user.

The phenomenon in a dream of the user who has a dream in the sleep state can be estimated based on image data corresponding to the biological information of the user. The degree of clarity of a phenomenon in a dream can be estimated based on, for example, edges, motion vector, contrast, and resolution with respect to the image data corresponding to the biological information of the user. The activity threshold value is set based on the level of the autonomic nervous system activity of the user. However, alternatively, the activity threshold value can be set based on the level of the image data corresponding to the biological information of the user.

In the first embodiment, the emotional level threshold value is set based on degree of variation in the emotions of the user. The human emotions include feelings such as joy, anger, sorrow, and pleasure, and, for example, can be divided into categories by specific emotions such as amazement, joy, anger, fear, sadness, and disgust. Hence, the emotional level threshold value can be set based on the degree of amazement, the degree of joy, the degree of anger, the degree of sadness, and the degree of disgust.

The controller 14E includes the biological information acquiring unit 51, an activity calculating unit 61, an emotional level calculating unit 62, a classifying unit 53E, and a processing unit 54E. The biological information acquiring unit 51 is identical to the fifth embodiment. The activity calculating unit 61 and the emotional level calculating unit 62 collectively function as the determining unit 52 according to the fifth embodiment.

The biological information acquiring unit 51 is connected to the measuring unit 12. The biological information acquiring unit 51 controls the measuring unit 12 and causes the measuring unit 12 to detect the biological information of the measuring unit 12. Then, the biological information acquiring unit 51 acquires the biological information of the user measured by the measuring unit 12.

The biological information acquiring unit 51 is connected to the activity calculating unit 61. The activity calculating unit 61 calculates the autonomic nervous system activity based on the biological signal acquired by the biological information acquiring unit 51. Regarding the calculation method implemented by the activity calculating unit 61 to calculate the autonomic nervous system activity, the explanation is given later.

The biological information acquiring unit 51 is connected to the emotional level calculating unit 62. The emotional level calculating unit 62 calculates the emotional level based on the biological signal acquired by the biological information acquiring unit 51. Regarding the calculation method implemented by the emotional level calculating unit 62 to calculate the emotional level, the explanation is given later.

The activity calculating unit 61 and the emotional level calculating unit 62 are connected to the memory 13E and the classifying unit 53E. The classifying unit 53E compares the autonomic nervous system activity which is calculated by the activity calculating unit 22 with an activity threshold value stored in the memory 13E to determine whether or not it is possible to perform the output operation. Moreover, the classifying unit 53E compares the emotional level which is calculated by the emotional level calculating unit 62 with an emotional level threshold value stored in the memory 13E to determine whether or not it is possible to perform the output process.

More particularly, when the user is in the sleep state and is having a dream and recalling a specific phenomenon in the dream (sensory information), the classifying unit 53E compares the autonomic nervous system activity at that time with the activity threshold value, and accordingly determines whether or not the output operation for outputting the phenomenon in the dream to the outside can be performed. In an identical manner, when the user is in the sleep state and is having a dream and recalling a specific phenomenon (sensory information), the classifying unit 53E compares the emotional level at that time with the emotional level threshold value, and accordingly determines whether or not the output operation for outputting the phenomenon in the dream to the outside can be performed.

In the sixth embodiment, when the autonomic nervous system activity of the user is equal to or lower than the activity threshold value, that is, when the user in the sleep state is having a low degree of clarity of the phenomenon in a dream of the user who has a dream, the classifying unit 53E turns off (OFF) a first flag that is set for disabling the output operation. Moreover, when the emotional level of the user is equal to or lower than the corresponding emotional level threshold value, that is, when the user in the sleep state is having a low degree of variation in the emotions felt toward the phenomenon in a dream of the user who has a dream in the sleep state, the classifying unit 53E turns off (OFF) a second flag that is set for disabling the output operation. When the first flag or the second flag is turned off (OFF), the classifying unit 53E allows implementation of the output operation for outputting the phenomenon in the dream to the outside, and classifies that the biological information of the user at that time can be output.

On the other hand, when the autonomic nervous system activity of the user is higher than the activity threshold value, that is, when the user in the sleep state is having a high degree of clarity of the phenomenon in a dream of the user who has a dream, the classifying unit 53E turns on (ON) the first flag that is set for disabling the output operation. Moreover, when the emotional level of the user is higher than the corresponding emotional level threshold value, that is, when the user in the sleep state is having a high degree of variation in the emotions felt toward the phenomenon in the dream of the user who has a dream, the classifying unit 53E turns on (ON) the second flag that is set for disabling the output operation. When the first flag and the second flag is turned on (ON), the classifying unit 53E disables implementation of the output operation for outputting the phenomenon in the dream to the outside, and classifies that the biological information of the user at that time cannot be output.

In that case, multiple relationships between the degree of clarity of the recalled phenomenon in the dream and the autonomic nervous system activity during the sleep state of the user are obtained in advance. Then, regarding the obtained relationships between the degree of clarity of the recalled phenomenon and the autonomic nervous system activity, it is desirable to set the activity threshold value according to the degree of clarity of the phenomenon in the dream which is acceptable to the user as an externally-outputtable phenomenon in the dream. Moreover, multiple emotional levels corresponding to the recalled phenomenon in the dream during the sleep state of the user are obtained in advance. Regarding the obtained emotional levels corresponding to the phenomenon in the dream, it is desirable to set the emotional level threshold value according to the emotional levels of the phenomenon in the dream which is acceptable to the user as an externally-outputtable phenomenon.

### Autonomic Nervous System Activity

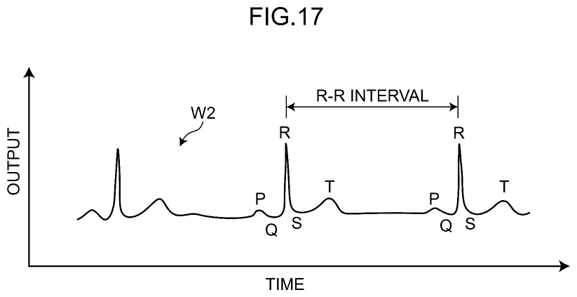

FIG. 16 is a graph for explaining physiological characteristics of a biological signal. FIG. 17 is a schematic diagram for explaining the autonomic nervous system activity. In the explanation of FIGS. 16 and 17, the biological signal is assumed to be electrocardiogram. However, instead of electrocardiogram, the biological signal such as a brain wave can be used. By a second-order differentiation of pulse waves, a signal corresponding to an R-R interval of electrocardiogram is able to be obtained.

As illustrated in FIG. 16, a waveform W1 representing electrocardiogram includes a P wave, a QRS wave, a T wave, and a U wave. The heart rate is measured by detecting the R wave which represents a peak of the QRS wave as one pulse.

The electrocardiogram is a waveform in which peaks called R-wave appear at regular time intervals. The pulse occurs due to autoignition of pacemaker cells in the sinoatrial node of the heart. Rhythm of the pulse is heavily influenced by the sympathetic nervous system and the parasympathetic nervous system. The sympathetic nervous system enhances the heart activity, while the parasympathetic nervous system suppresses the heart activity. Normally, the sympathetic nervous system and the parasympathetic nervous system act to counterbalance each other. When at rest or in a state close to resting, the parasympathetic nervous system becomes dominant. Normally, when adrenaline is secreted due to the activation of the sympathetic nervous system, the pulse rate increases. On the other hand, when acetylcholine is secreted due to the activation of the parasympathetic nervous system, the pulse rate decreases. Hence, regarding a functional inspection of the autonomic nerve system, it is assumed that checking variability in an R-R interval in the electrocardiogram proves useful.

As illustrated in FIG. 17, in a waveform W2 representing the electrocardiogram, the R-R interval indicates an interval between chronologically continuous R-wave. The heart rate variability is measured by treating the R wave, which represents the peak of the QRS wave, as one pulse. The variability in the interval between the R waves in the electrocardiogram, that is, a fluctuation in the time interval of the R-R interval in FIG. 3 is used as an autonomic nervous system indicator. An appropriateness of using the fluctuation in the time interval of the R-R interval as the autonomic nervous system indicator has been reported in many medical institutions. The fluctuation of the R-R interval increases when at rest and decreases when in stress.

The variability in the R-R interval includes a few types of characteristic fluctuations. One type of fluctuation represents low-frequency component appearing in the vicinity of 1 Hz and is attributed to the variation in the sympathetic nervous system along with the blood pressure feedback control of the blood vessels. Another type of fluctuation indicates the variation occurring in synchronization with breathing, and represents high-frequency component that reflect the respiratory sinus arrhythmia. The high-frequency component reflects direct interference with vagal preganglionic neuron due to the respiratory center, stretch receptor reflex, and baroreceptor reflex of the blood pressure change due to the breathing, and is treated as the parasympathetic nervous system indicator that mainly affects the heart. That is, it can be said that, from among waveform components in which the fluctuation between the R-R waves of the electrocardiogram is measured, power spectrum of the low-frequency component represents the activity of the sympathetic nervous system, and power spectrum of the high-frequency components represent the activity of the parasympathetic nervous system.

The fluctuation of the input electrocardiogram is obtained from a differential value of the R-R interval value. In that case, when the differential values of the R-R intervals do not represent equally spaced time-series data, the activity calculating unit 61 converts those values into equally spaced time-series data using a three-dimensional spline interpolation. The activity calculating unit 61 performs orthogonal transform such as fast Fourier transform with respect to the differential values of the R-R intervals. Thus, the activity calculating unit 61 calculates the power spectrum of the high-frequency components and the power spectrum of the low-frequency components of the differential values of the R-R interval values of the electrocardiogram. The activity calculating unit 61 calculates a sum total of the power spectrum of the high-frequency component as RRHF. Moreover, the activity calculating unit 61 calculates a sum total of the power spectrum of the low-frequency component as RRLF. The activity calculating unit 61 calculates the autonomic nervous system activity using the following equation.

AN=(C1+RRLF)/(C1+RRHF)+C2

In the equation given above, AN represents the autonomic nervous system activity, RRHF represents the sum total of the power spectrum of the high-frequency component, and RRLF represents the sum total of the power spectrum of the low-frequency component. Moreover, C1 and C2 are fixed values defined for suppressing divergence of solutions of the autonomic nervous system activity AN.

The activity calculating unit 61 sets an activity threshold value based on the multiple autonomic nerve activities AN calculated for the user, and stores the activity threshold value in the memory 13E.

### Emotional Level

In an identical manner to the autonomic nervous system activity, the emotional level is also calculated based on a biological signal such as the electrocardiogram or the brain wave signal acquired from the user. For example, the emotional level calculating unit 62 calculates the emotional level based on the brain wave signal acquired from the user. More particularly, the emotional level calculating unit 62 extracts α waves and β waves from the brain wave signals. The α waves increase in a relaxed state, and the β waves increase when joy, anger, or nervousness is felt. Hence, the emotional level calculating unit 62 calculates the emotional level according to the extracted β waves/α waves. However, the abovementioned calculation method for calculating the emotional level is only exemplary. Thus, the emotional level calculating unit 62 can calculate the emotional level using the electrocardiogram from among the biological signals, or can calculate the emotional level using the electrocardiogram and the brain wave signals from among the biological signals.

In the memory 13E, the emotional level threshold value is stored in advance. Since the emotional level threshold value is different for each individual, the emotional level can be calculated according to the calculation method explained above for calculating the emotional level, and the emotional level threshold value can be set with reference to the values thereof.

### Information Processing Method

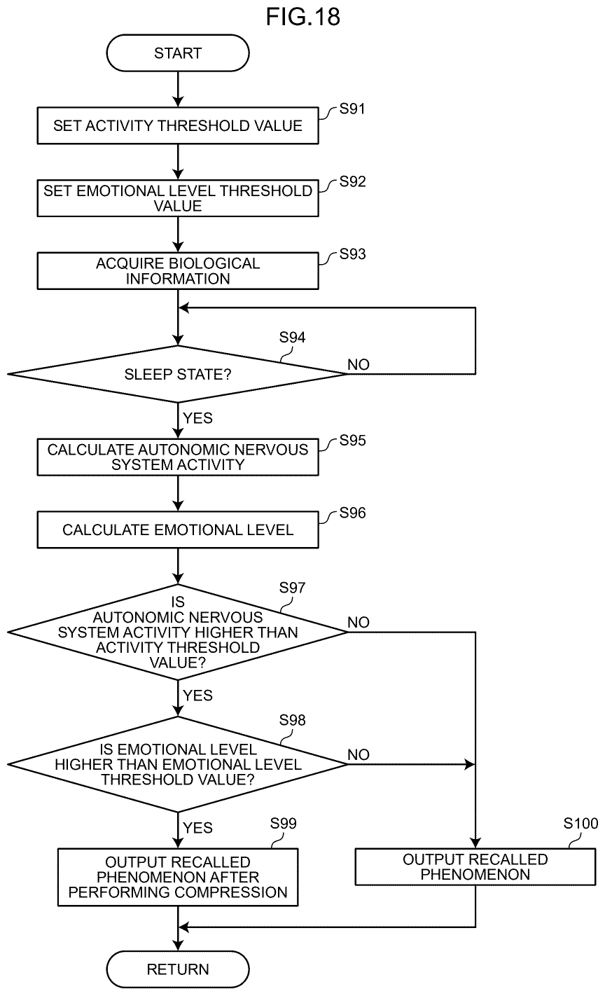

FIG. 18 is a flowchart for explaining an information processing method according to the sixth embodiment.

As illustrated in FIGS. 15 and 18, at Step S91, the activity threshold value is set. It is desirable to set the activity threshold value separately for each user of the information processing device 10E. At Step S12, the emotional level threshold value is set. It is desirable to set the emotional level threshold value separately for each user of the information processing device 10E. The processes at Steps S91 and S92 can be performed before the information processing device 10E is used.

After the operations at Steps S91 and S92 are completed, the user of the information processing device 10E goes to sleep. At Step S93, the measuring unit 12 measures the biological information of the user, and the biological information acquiring unit 51 acquires the biological information of the user measured by the measuring unit 12. At Step S94, based on the biological information of the user, the controller 14E determines whether or not the user is in the sleep state. According to the biological information of the user, when the autonomic nervous system activity calculated using the electrocardiogram is determined to match with the autonomic nervous system activity during REM sleep or during non-REM sleep, the controller 14E determines that the user has transitioned into the sleep state. For example, the controller 14E can determine that the user has transitioned into the sleep state based on the brain waves, the electrocardiogram, the pulse waves, the pulse rate, the respiratory rate, and the autonomic nervous system activity as the biological information. At Step S95, based on the biological information of the user, the controller 14E can determine whether or not the user is in the REM sleep state or in the non-REM sleep state.

When it is determined that the user is not in the sleep state (No), the controller 14E maintains the present state. On the other hand, when it is determined that the user is in the sleep state (Yes), at Step S95, the activity calculating unit 61 calculates the autonomic nervous system activity based on the biological information of the user acquired by the biological information acquiring unit 51. At Step S96, the emotional level calculating unit 62 calculates the emotional level based on the biological information of the user acquired by the biological information acquiring unit 51. Meanwhile, the process performed by the activity calculating unit 61 and the process performed by the emotional level calculating unit 62 can be performed in reverse order or in a simultaneous manner. At Step S97, the classifying unit 53E determines whether or not the autonomic nervous system activity which is calculated by the activity calculating unit 61 is higher than the activity threshold value of the user stored in the memory 13E. When the classifying unit 53E determines that the autonomic nervous system activity of the user is equal to or lower than the corresponding activity threshold value (No), the process proceeds to Step S100.

On the other hand, when the classifying unit 53E determines that the autonomic nervous system activity of the user is higher than the corresponding activity threshold value (Yes), the process proceeds to Step S98. At Step S98, the classifying unit 53E determines whether or not the emotional level calculated by the emotional level calculating unit 62 is higher than the corresponding emotional level threshold value for the user stored in the memory 13E. When the classifying unit 53E determines that the emotional level of the user is equal to or lower than the corresponding emotional level threshold value (No), the process proceeds to Step S100. On the other hand, when the classifying unit 53E determines that the emotional level of the user is higher than the corresponding emotional level threshold value (Yes), the process proceeds to Step S99.

That is, when it is determined at Step S97 that the autonomic nervous system activity of the user is equal to or lower than the corresponding activity threshold value and when it is determined at Step S98 that the emotional level of the user is equal to or lower than the corresponding emotional level threshold value, the classifying unit 53E classifies that the biological information of the user or the phenomenon recalled by the user can be output without modification. Then, the system control proceeds to Step S100, and the output unit 15 outputs, without modification, the biological information of the user to the outside, that is, outputs, without modification, the phenomenon recalled by the user to the outside. On the other hand, when it is determined at Step S97 that the autonomic nervous system activity of the user is higher than the corresponding activity threshold value and when it is determined at Step S98 that the emotional level of the user is higher than the corresponding emotional level threshold value, the classifying unit 53E classifies that the biological information of the user or the recalled phenomenon can be output after the processing unit 54E has compressed the biological information of the user or the recalled phenomenon. Then, process proceeds to Step S99, and the output unit 15 outputs the biological information, which has been encrypted or encoded, to the outside, that is, outputs the phenomenon recalled by the user, which have been compressed, to the outside.

### Effects of Embodiments

The information processing devices according to the embodiments include the biological information acquiring unit 21 configured to acquire the biological information of the user (subject); the activity calculating unit 22 configured to calculate the activity of the user based on the biological information acquired by the biological information acquiring unit 21; the emotional level calculating unit 23 configured to calculate the emotional level of the user based on the biological information acquired by the biological information acquiring unit 21; and the classifying unit 24 configured to classify the biological information based on the activity which is calculated by the activity calculating unit 22 and the emotional level which is calculated by the emotional level calculating unit 23.

Thus, as a result of classifying the biological information of the user based on the activity and the emotional level of the user, it becomes possible to ensure security for outputting, to the outside, the phenomenon recalled by the user in the sleep state, and to enable protection of the phenomenon recalled by the subject in the sleep state.

The information processing devices according to the embodiments include the memory 13 in which the activity threshold value which is set in advance and the emotional level threshold value which is set in advance are stored, and the classifying unit 24 is further configured to classify, when the activity is equal to or lower than the activity threshold value or when the emotional level is equal to or lower than the emotional level threshold value, that the output information can be output. Thus, the classification performed by the classifying unit 24 can be implemented in an appropriate manner.

The information processing devices according to the embodiments include further includes a processing unit 25 configured to encrypt or encode the biological information when the classifying unit classifies that the activity is higher than the activity threshold value and the emotional level is higher than the emotional level threshold value. Hence, it becomes possible to ensure security for outputting, to the outside, the phenomenon recalled by the user in the sleep state.

The information processing devices according to the embodiments include the determining unit 32 configured to determine the sleep state of the user based on the biological information acquired by the biological information acquiring unit 21; the classifying units 33 or 33B configured to classify the biological information based on the sleep state of the user determined by the determining unit 32; and the processing unit 34 or 34B configured to encrypt the biological information when the classifying unit 33 or 33B classifies that the biological information indicates the REM sleep state.

Thus, as a result of classifying the biological information of the user based on the sleep state of the user, it becomes possible to ensure security for outputting, to the outside, the phenomenon recalled by the user in the sleep state, and to enable protection of the phenomenon recalled by the subject in the sleep state.

The information processing devices according to the embodiments include the memory 13A in which the activity threshold value which is set in advance and the emotional level threshold value which is set in advance are stored, and the processing unit 34B is further configured to encrypt the biological information when the classifying unit 33B classifies that the activity is higher than the activity threshold value and that the emotional level is higher than the emotional level threshold value Thus, the classification performed by the classifying unit 33B can be implemented in an appropriate manner.

The information processing devices according to the embodiments include the determining unit 52 configured to determine the sleep state of the user based on the biological information acquired by the biological information acquiring unit 51; the classifying unit 53 or 53E configured to classify the biological information based on the sleep state of the user determined by the determining unit 52; and the processing unit 54 or 54E configured to compress the biological information when the classifying unit 53 or 53B classifies that the biological information indicates the REM sleep state.

Thus, as a result of classifying the biological information of the user based on the sleep state of the user, it becomes possible to ensure security for outputting, to the outside, the phenomenon recalled by the user in the sleep state, and to enable protection of the phenomenon recalled by the subject in the sleep state.

The information processing devices according to the embodiments include the memory 13A in which the activity threshold value which is set in advance and the emotional level threshold value which is set in advance are stored, and the processing unit 54E is further configured to compress the biological information when the classifying unit 53 or 53E classifies that the activity is higher than the activity threshold value and that the emotional level is higher than the emotional level threshold value. Thus, the classification performed by the classifying units 53 and 53E can be implemented in an appropriate manner.

In the above description, the explanation was given about the information processing device according to the present invention. However, the present application can be implemented according to various other forms other than the embodiments described above.

The constituent elements of the guidance device illustrated in the drawings are merely conceptual, and need not be physically configured as illustrated. The constituent elements, as a whole or in part, can be separated or integrated either functionally or physically based on various types of loads or use conditions.

The information processing device is configured using, for example, a program that is loaded as software in a memory. In the embodiments described above, the configuration is explained with reference to function blocks implemented as a result of coordination between hardware and software. Such function blocks can be implemented in various ways, such as using only hardware, or using only software, or using a combination of hardware and software.

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

The information processing device, the information processing method, and the non-transitory storage medium according to the present application can be applied to a technology for controlling the brain activity of the user.

According to the present application, it becomes possible to enable protection of the phenomenon recalled by the subject in the sleep state.

What is claimed is:

1. An information processing device comprising:
a biological information acquiring unit configured to acquire biological information of a subject;
an activity calculating unit configured to calculate activity of the subject based on the biological information acquired by the biological information acquiring unit;
an emotional level calculating unit configured to calculate emotional level of the subject based on the biological information acquired by the biological information acquiring unit; and
a classifying unit configured to classify the biological information based on the activity which is calculated by the activity calculating unit and the emotional level which is calculated by the emotional level calculating unit.

2. The information processing device according to claim 1, further comprising a memory in which an activity threshold value which is set in advance and an emotional level threshold value which is set in advance are stored, wherein
the classifying unit is further configured to classify, when the activity is equal to or lower than the activity threshold value or when the emotional level is equal to or lower than the emotional level threshold value, that the biological information is able to be output.

3. The information processing device according to claim 2, further comprising a processing unit configured to encrypt or encode the biological information when the classifying unit classifies that the activity is higher than the activity threshold value and the emotional level is higher than the emotional level threshold value.

4. The information processing device according to claim 1, further comprising:
a determining unit configured to determine a sleep state of the subject based on the biological information acquired by the biological information acquiring unit;
a classifying unit configured to classify the biological information based on the sleep state of the subject determined by the determining unit; and
a processing unit configured to encrypt the biological information when the classifying unit classifies that the biological information indicates a REM sleep state.

5. The information processing device according to claim 4, further comprising a memory in which an activity threshold value which is set in advance and an emotional level threshold value which is set in advance are stored, wherein
the processing unit is further configured to encrypts the biological information when the classifying unit classifies that the activity is higher than the activity threshold value and the emotional level is higher than the emotional level threshold value.

6. The information processing device according to claim 1, further comprising:
a determining unit configured to determine a sleep state of the subject based on the biological information acquired by the biological information acquiring unit;
a classifying unit configured to classify the biological information based on the sleep state of the subject determined by the determining unit; and
a processing unit configured to compress the biological information when the classifying unit classifies that the biological information indicates a REM sleep state.

7. The information processing device according to claim 6, further comprising a memory in which an activity threshold value which is set in advance and an emotional level threshold value, which is set in advance are stored, wherein
the processing unit is further configured to compress the biological information when the classifying unit classifies that the activity is higher than the activity threshold value and the emotional level is higher than the emotional level threshold value.

8. An information processing method comprising:
acquiring biological information of a subject;
calculating activity of the subject based on the biological information;
calculating emotional level of the subject based on the biological information; and
classifying the biological information based on the activity and the emotional level.

9. A non-transitory storage medium that stores a program that causes a computer, which operates as an information processing device, to execute:
acquiring biological information of a subject;
calculating activity of the subject based on the biological information;
calculating emotional level of the subject based on the biological information; and
classifying the biological information based on the activity and the emotional level.
